(19) United States
(12) Patent Application Publication
(10) Pub. No.: US 2026/0242855 A1
(43) Pub. Date: Aug. 20, 2026

(54) METHOD FOR SPATIAL TRACING AND SEQUENCING OF CELLS OR ORGANELLES

(71) Applicants: PARIS SCIENCES ET LETTRES, PARIS (FR); CENTRE NATIONAL DE LA RECHERCHE SCIENTIFIQUE, PARIS (FR); ECOLE SUPERIEURE DE PHYSIQUE ET DE CHIMIE INDUSTRIELLES DE LA VILLE DE PARIS, PARIS (FR)

(72) Inventors: Gaël BLIVET-BAILLY, PARIS (FR); Andrew GRIFFITHS, PARIS (FR); Pablo IBANEZ, ARGENTEUIL (FR); Phillipe NGHE, SAINT-MANDE (FR); Yannick RONDELEZ, PARIS (FR)

(21) Appl. No.: 18/992,813

(22) PCT Filed: Jul. 12, 2023
(86) PCT No.: PCT/EP2023/069282

§ 371 Date: Jan. 9, 2025

(30) Foreign Application Priority Data
Jul. 12, 2022 (EP) 22306049.2

(51) Int. Cl. C12Q 1/6841

(52) U.S. Cl. CPC C12Q 1/6841

(57) ABSTRACT

The present invention concerns a labelling solution on or into a biological sample which allows identification tags to be assigned to areas of the biological sample comprising a single or plurality of cells or organelles, e.g. about two to ten cells.

The present invention concerns a labelling solution on or into a biological sample which allows identification tags to be assigned to areas of the biological sample comprising a single or plurality of cells or organelles, e.g. about two to ten cells.

### TECHNICAL BACKGROUND

Current sequencing techniques of single cells are very powerful because they allow, for example, access to “omic” information (genomic, epigenomic transcriptomic, proteomic, etc.) for each of the cells in a sample, but they require the dissociation of the cells leading to the loss of localisation information for each of the cells in the starting sample.

Indeed, a major issue e.g., in oncology is how to accurately characterize the tumour/microenvironment interactions, which requires access to both molecular signals and the intra-tissue spatial localisation of the cells that exchange these signals. These interactions may play a major role in the survival or death of tumour cells. A better understanding of these interactions would help to overcome the adaptive mechanisms taking place in the tumour system.

Although “omics” technologies have revolutionized molecular biology by making genomic, transcriptomic, epigenomic and proteomic analyses possible on a very large scale, they have, until recently, only yielded “average” profiles of multiple cells that do not take into account the cellular heterogeneity present in healthy and pathological tissues.

Recently, a number of technologies for single cell analysis has been developed. In particular, droplet microfluidic systems in which individual cells are co-encapsulated in droplets with beads harbouring barcoded primers to allow for sequencing of RNA at the single-cell level (scRNA-seq) from several thousands of cells. However, in these systems, the cells in the tissue are dissociated prior to analysis and there is no correlation between the single cell sequencing data and the localisation of these same cells in the original tissue.

Single-cell analysis technologies allowing spatial localisation of the measured signals are currently very limited, both in terms of number of targets (immunohistochemistry, in situ RNA hybridization) and number of cells (e.g. sequencing of individual cells after laser capture microdissection (LCM)). Although “spatial transcriptomics” (Vickovic, Sanja, et al. Nature methods 16.10 (2019): 987-990.), “Slide-seq” (Rodriques, Samuel G., et al. Science 363.6434 (2019): 1463-1467) and “DNA nanoball stereo-sequencing” (Chen et al., <<Large Field of View-Spatially Resolved Transcriptomics at Nanoscale Resolution>> bioRxiv 2021.01.17.427004) systems allow spatial resolution in individual tissue sections, they do not provide access to single cell data. “XYZ-seq” (Lee et al., Sci Adv. 2021 Apr. 21; 7(17):eabg4755) measure genome-wide single-cell expression but at low spatial resolution (500 μm resolution) with less mRNA reading and less detected genes than standard scRNA-seq. Fluorescence in situ sequencing (FISSEQ) uses in situ sequencing to spatially localise the expression of multiple genes in fixed tissues, with a short reading (30 bases) and with only about 200 mRNA readings per cell (compared to about 40,000 in scRNA-seq). Digital Spatial Profiler (DSP) is a platform developed by Nanostring based on the sequencing of photocleavable oligonucleotide markers released from a targeted tissue area by UV exposure. Data on the localisation of cells in the tissue provide a numerical and spatial profile of RNA or target abundance. However, this technique does not provide access to the complete transcriptome, does not have unicellular resolution, and allows analysis of only a small number of areas.

In summary, the current technological tools to study cellular interactions in tissues, interactions at the single-cell level, are still limited because they only allow spatial localisation of signals from a limited number of molecular targets for a large number of cells, or measurement of a large number of molecular targets for a limited number of localised (microdissected) cells, or measurement of a large number of molecular targets on thousands of non-localised cells.

Currently, the coupling, for thousands of individual cells, of (i) intra-tissue spatial localisation information of each cell to (ii) the measurement of molecular signals from each of these same cells is clearly identified as a major technological challenge and as a clinical need in order to better understand, for example, the adaptation mechanisms occurring in tumours and to adjust patient treatment accordingly.

The methods and kits provided by the invention fulfil this need for a system allowing single-cell “omics” analysis of thousands of cells with a spatial resolution of a few dozen cells, or less, from a tissue.

### SUMMARY OF THE INVENTION

The invention relates to a method for labelling individual cells or organelles within a biological sample with an identification nucleic acid sequence, the method comprising:
a) providing a first set of nucleic acids (“Emitter nucleic acids”) wherein each nucleic acid molecule comprises an amplification sequence, an identification sequence, and a capture sequence;
b) providing a second set of nucleic acids (“Receptor nucleic acids”), wherein each Receptor nucleic acid is coupled, covalently or non-covalently, to a ligand of a cell target or organelle target, wherein the Receptor nucleic acid comprises i) an amplification sequence matching all or part of the amplification sequence of the set of Emitter nucleic acids, or a complement thereof, or ii) a capture sequence matching all or part of the capture sequence of the set of Emitter nucleic acids, or a complement thereof;
c) contacting the sets of Emitter nucleic acids and Receptor nucleic acids, in solution, with a biological sample so as to label the individual cells or organelles within the biological sample at least with the Receptor nucleic acids;
d) releasing multiple nucleic acid molecules that are copies of the region of the Emitter nucleic acids that comprises the amplification sequence, the identification sequence, and the capture sequence, or the reverse complement thereof (“Emitted nucleic acids”), and hybridizing the Emitted nucleic acids to Receptor nucleic acids;
e) dissociating the biological sample and recovering individualized cells or organelles, wherein at least a sub-population of the individualised cells or organelles is labelled with the Emitted nucleic acids.

In a second aspect, the invention relates to a method of mapping and sequencing individual cells or organelles of a biological sample, the method comprising:
a) Providing individualized cells labelled with (i) a nucleic acid that comprises an amplification sequence, an identification sequence, and a capture sequence, or (ii) the reverse complement thereof (i), as obtainable by the method for labelling individual cells or organelles according to the invention;
b) Trapping the individualized cells or organelles labelled with said nucleic acid or the reverse complement thereof in a compartment, wherein the compartment comprises a compartment-specific nucleic acid and at least one of the following sequences for nucleic acid labeling and further sequencing: hybridization site, ligation site or recombination site;
c) Optionally analysing captured cells, organelles and/or molecules they secrete, using optical detection;
d) Lysing trapped cells, or cells and organelles, thereby releasing nucleic acids from the cells or organelles in the compartments;
e) Associating i) the compartment-specific sequence with ii) the nucleic acids released from the cells or organelles in the compartments and iii) the nucleic acids comprising the amplification sequence, the identification sequence, and the capture sequence, or the reverse complement thereof;
f) Recovering the nucleic acids produced at step e) from the compartments and sequencing recovered nucleic acids; and
g) Defining nucleic acids comprising the same compartment-specific sequence as originating from the same single cell, and mapping the position of the single cell originally on the biological sample based on the identification sequence(s), or reverse complementary sequence thereof, contained in the nucleic acids produced at step e), thereby combining mapping and sequencing information of the individual cells of the biological sample;
h) Mapping of the position of the single cell originally on the biological sample by determining the relative proportions of identical identification sequence(s), or reverse complementary sequence thereof, contained in the nucleic acids produced at step e) to estimate the distance between cells, on the principle of triangulation; and
i) Optionally, mapping the sequencing information back onto an image from microscopy of the biological sample, taken before dissociation.

In a third aspect, the invention relates to a kit comprising:
a) Emitter nucleic acids wherein each emitter nucleic acid comprises an amplification sequence, an identification sequence, and a capture sequence;
b) Receptor nucleic acids wherein each receptor nucleic acid comprises i) an amplification sequence matching all or part of the amplification sequence of the set of Emitter nucleic acids, or a complement thereof, or ii) a capture sequence matching all or part of the capture sequence of the set of Emitter nucleic acids, or a complement thereof;
c) Ligands of a cell target or organelle target;
d) Optionally a nicking endonuclease and a polymerase with strand displacement activity.

### DETAILED DESCRIPTION

The present invention concerns a solution to assign identification nucleic acids by diffusion to areas of a biological sample, such as a tissue section, comprising a plurality of cells or organelles through cells (optionally permeabilized), e.g. about two to ten cells. As cells carrying the same identification nucleic acid are in the same area, it allows the reconstruction of the network of neighbouring cells after dissociation of the biological sample, and single-cell analysis of the cellular nucleic acids, and therefore reconstruct the spatial organisation of cells prior to dissociation. Different proportions of specific identification nucleic acids allow to estimate the original distance between cells, based on the principle of triangulation. The present invention relies on local isothermal amplification or pre-amplification of identification nucleic acid sequences followed by their diffusion. The identification nucleic acid sequences can be associated to the surrounding cells via a ligand modified to conjugate with the produced nucleic acids.

The invention thus makes it possible for cells of a biological sample to be dissociated, analysed and sequenced individually (single cell) using current techniques (droplet microfluidics, valve microfluidics, microplates, thermoactuable hydrogels), with the possibility for each cell to determine its position in the starting biological sample.

### Method for Labelling Individual Cells or Organelles

In a first aspect, it is provided a method for labelling individual cells or organelles within a biological sample with an identification nucleic acid sequence.

In a first embodiment, a solution comprising ligands carrying a first set of nucleic acids called “Emitter nucleic acids” that comprise an identification sequence, and ligands carrying a second set of nucleic acids called “Receptor nucleic acids” are put in contact with a biological sample. After ligand conjugation to the cells or organelles, the biological sample is rinsed before adding a second solution at the surface of the tissue containing a nicking endonuclease, a polymerase with strand displacement activity and, optionally, additional nucleic acids to improve local amplification in a buffer compatible with enzymes activity. By the action of endonuclease and polymerase on the Emitter nucleic acids, copies of a part of these nucleic acids comprising the identification sequence will be released. Copies of these “Emitted nucleic acids” that comprise an identification sequence will then be captured on “Receptor” ligands by hybridization on the Receptor nucleic acids.

In a second embodiment, Emitter nucleic acids containing identification sequences are amplified using Rolling Circle Amplification (RCA), to form concatemers of identical nucleic acids before being put in contact with a biological sample with a restriction enzyme to break the concatemers. Ligands carrying Receptor nucleic acids are also put in contact with the biological sample. Cleaved concatemer fragments become associated with the cells or organelles by hybridization on the Receptor nucleic acids. As an alternative, in this second embodiment, nucleic acids containing identification sequences can be provided in the form of beads bearing multiple Emitter nucleic acids that comprise an identical identification sequence.

As used herein, the biological sample encompasses any network of neighboring cells, such as a tissue sample, an organoid, a 2D cell culture (in particular 2D confluent cell culture), or a 3D cell culture. The biological sample can be either fresh, frozen or fixed. According to an embodiment, the biological sample is a tumour tissue sample.

In the frame of the method, a first set of nucleic acids (“Emitter nucleic acids”) is provided wherein each nucleic acid molecule comprises, consecutively, an amplification sequence, an identification sequence, and a capture sequence, preferably in the 5′-3′ direction.

At least part of the nucleic acid molecules of the set of Emitter nucleic acids differ from the other nucleic acid molecules by the identification sequence, at least. According to an embodiment, each nucleic acid molecule, of the set of Emitter nucleic acids differ from the other nucleic acid molecules by the identification sequence, at least. The different identification sequences can be barcode sequences, or comprise both constant sequence(s) and a barcode sequence. By “barcode sequence”, it is meant a nucleic acid sequence which is random or which presents a defined (i.e. known) sequence.

According to some embodiments, the amplification sequence is identical in all or part (preferably all) of the nucleic acid molecules of the set of Emitter nucleic acids.

According to some embodiments, the capture sequence is identical in all or part (preferably all) of the nucleic acid molecules of the set of Emitter nucleic acids.

The Emitter nucleic acids are preferably at most 110, 100, 90, 80, 70 or preferably 60 nucleotide long, but longer sequences can also be used. Typically, each of the amplification sequence, identification sequence, and capture sequence is 10 to 30 nucleotides long, preferably 12 to 25 nucleotide long.

In some embodiments, the Emitter nucleic acids further comprise a nicking site (for the nicking endonuclease), as well as, some consecutive nucleotides (forming a priming site) adjacent to the nicking site, to allow priming for amplification through polymerisation and nicking.

The Emitter nucleic acids are preferably DNA molecules, and may be single stranded DNA or partially double stranded DNA (hairpin DNA).

For example, the Emitter nucleic acid comprises or consists of

(SEQ ID NO: 2)
AACACCAAACCCTTCTAAAGCCCAAACCTCNNNNNNNNNNNNAAGCGATCTGTTACC
AAGCCGT CCTCAGC AGGATTAGAG TTTTT CTCTAATCCTGC,

wherein the underlined nucleotides represent the capture sequence, the nucleotides in italic a barcode sequence which is the identification sequence, and the nucleotides in bold represent the amplification sequence together with a nicking site, a priming site, a loop and the reverse complement of the priming site (for self-priming).

According to the first embodiment of the method, each Emitter nucleic acid is bound to a ligand of a cell target or organelle target.

According to this embodiment, the Emitter nucleic acids additionally comprise a sequence complementary to a nicking site, in 3′ of the amplification sequence.

According to the second embodiment of the method, the set of Emitter nucleic acids is provided in the form of group(s) of Emitter nucleic acids wherein all Emitter nucleic acids of a group comprise an identical identification sequence. According to this embodiment, Emitter nucleic acids are not bound to a ligand of a cell target or organelle target.

In particular, the groups of Emitter nucleic acids may be provided in the form of concatemers of Emitter nucleic acids generated by rolling-circle replication, wherein each concatemer comprises identical Emitter nucleic acids. The groups of Emitter nucleic acids may also be provided in the form of beads carrying Emitter nucleic acids that comprise an identical identification sequence or beads carrying identical Emitter nucleic acids.

In this second embodiment, the sequence of the Emitter nucleic acids then comprises a restriction site or a cleavage site (such as a photocleavage, a diol linkage, a peptide linkage or via a modified base such as uracil, methylated or RNA), in order to release from the concatemers or beads a region of the Emitter nucleic acids that comprises the amplification sequence, the identification sequence, and the capture sequence, notably under the action of a restriction enzyme in the case of a restriction site.

In the frame of the method, a second set of nucleic acids (“Receptor nucleic acids”) is provided wherein each Receptor nucleic acid is coupled, covalently or non-covalently, to a ligand of a cell target or organelle target.

A Receptor nucleic acid is designed to comprise i) an amplification sequence matching all or part of an amplification sequence of the set of Emitter nucleic acids, or a complement thereof, or ii) a capture sequence matching all or part of a capture sequence of the set of Emitter nucleic acids, or a complement thereof.

As used herein the term “matching” indicates that a sequence has complete identity with another sequence over a stretch of contiguous nucleotides, preferably over at least 8, 10, 12, 14, 16, or more contiguous nucleotides, and still preferably over the total length of the shorter of the two sequences.

According to some embodiments, the Receptor nucleic acid further comprises a restriction site (for an endonuclease) or a cleavage site (such as a photocleavage, a diol linkage, a peptide linkage or via a modified base such as uracil, methylated or RNA) in order for the Receptor nucleic acid to be able to release the captured identification sequences at the appropriate time, for example after isolation in a compartment for single-cell barcoding.

For instance, a Receptor nucleic acid may comprise or consist of AACACCAAACCCTTCTAAAGCCCAAACCTC (SEQ ID NO:3, i.e. may comprise or consist of the capture sequence of the Emitter nucleic acid of sequence SEQ ID NO:2) or ACACGTTGATCTAGTCGCCACCAACACCAAACCCTTCTAAAGCCCAAACCTCCATTC GTTCCGCTCGCAACAAT (SEQ ID NO:4) when the Emitter nucleic acid comprises or consists of SEQ ID NO:2. In SEQ ID NO:4 wherein the nucleotides in bold represent a sequence to hybridise a quencher, the stretch of nucleotides in standard characters consists of SEQ ID NO:3, and the underlined nucleotides represent a spacer with the biotin.

The Receptor nucleic acids are typically, 10 to 30 nucleotides long, preferably 12 to 25 nucleotide long.

The Receptor nucleic acids are preferably DNA molecules, and still preferably single stranded DNA. Receptor nucleic acids may also be double stranded and partially single-stranded DNA, to allow ligation with emitted sequences.

The ligand binds to a cell target or organelle target in the biological sample. According to an embodiment, the ligand binds to a receptor or receptors at the surface or inside cells. According to another embodiment, the ligand binds to a receptor or receptors at the surface of, or inside, an organelle of cells. As used herein an organelle includes, without limitation, mitochondria, chloroplast, endoplasmic reticulum, flagellum, Golgi apparatus, nucleus, and vacuole.

For instance, the target present at the surface or inside the cells or organelles is selected from the group consisting of a cell or organelles surface protein (e.g. CD45, CD3, CD19, CD98, CD298, β2 microglobulin), a carbohydrate (e.g. Mannose, Galactose, N-acetylglucosamine), and a component of the lipid bilayer of cells or organelles.

Preferably the ligand is selected from the group consisting of an antibody, an aptamer, a lectin, and a peptide.

According to an embodiment, the cell target or organelle target bound by the ligand is a target ubiquitously present at the surface or inside all or most of the cells or organelles of the cells in the biological sample (e.g. CD98, CD298, β2 microglobulin, a lectin, Mannose, Galactose, lipid bilayer).

According to another embodiment, the cell target or organelle target bound by the ligand is present inside or at the surface of only a sub-set of the cells or organelles in the tissue sample (e.g. CD45, CD3, CD19).

The ligands of the Receptor nucleic acids may be all identical, or different, preferably all identical.

The ligands of the Emitter nucleic acids may be all identical, or different, preferably all identical.

In an embodiment, the ligands of the Emitter nucleic acids and Receptor nucleic acids are identical.

According to the method for labelling individual cells or organelles, the sets of Emitter nucleic acids and Receptor nucleic acids are contacted, in solution, with a biological sample so as to label the individual cells or organelles within the biological sample with at least the Receptor nucleic acids.

In the case of organelle analysis, the biological sample (e.g. tissue sample) may need to be permeabilized to allow copies of the nucleic acids to diffuse through the permeabilized cells and reach the organelles. Alternative methods known to the skilled person may be employed to make it possible for the nucleic acids to reach the organelles, such as using beads carrying emitter nucleic acids with a penetrating peptide at one end, or antibody specific for cell surface receptors that induce receptor-mediated endocytosis when they bind, leading to the internalisation of the antibody.

According to the first embodiment of the method wherein each Emitter nucleic acid is bound to a ligand of a cell target or organelle target, both the Receptor nucleic acids and Emitter nucleic acids label the cells or organelles of the biological sample.

In this first embodiment of the method, production of Emitted nucleic acids that comprise the reverse complement of the amplification sequence, the identification sequence, and the capture sequence, is performed by isothermal amplification of the Emitter nucleic acids by nicking and polymerization, catalysed by a nicking endonuclease and a polymerase with strand displacement activity. Accordingly, step c) of the method further comprise adding a nicking endonuclease and a polymerase with strand displacement activity to the solution that comprise the sets of Emitter nucleic acids and Receptor nucleic acids, and that is contacted with the biological sample. The 3′ part of the Emitter nucleic acids may be directly self-primed for polymerization, or, to improve local amplification, may be primed by addition of a free oligonucleotide that hybridizes to the 3′ part of the Emitter nucleic acids.

According to the second embodiment of the method wherein the set of Emitter nucleic acids is provided in the form of groups of Emitter nucleic acids having identical identification sequence, only the Receptor nucleic acids label the cells or organelles of the biological sample.

In this second embodiment, Emitter nucleic acids of a group (concatemer or bead) are cut by a restriction endonuclease to release multiple copies of a region of the Emitter nucleic acids that comprises the amplification sequence, the identification sequence, and the capture sequence (“Emitted nucleic acids”).

In both embodiments of the method, the biological sample is typically incubated between 25° C. and 37° C. for 10 to 60 min together with the Emitter nucleic acids, Receptor nucleic acids, and (i) nicking endonuclease and a polymerase or (ii) restriction endonuclease, as appropriate. The solution comprises a buffer compatible with enzyme activity. In the case of the presence of a cleavage site (such as a photocleavage, a diol linkage, a peptide linkage or via a modified base such as uracil, methylated or RNA, as described above), the biological sample is typically exposed to UV rays, or to an adapted chemical agent that is able to break said linkage.

After contacting/incubation, the biological sample is preferably rinsed.

In some embodiments, an image of the biological sample is taken by microscopy, optionally fluorescent microscopy, before dissociation. A labeling, such as fluorescent labelling, of the biological sample can be done before, at the same time, or even after the labelling with the Receptor nucleic acids and Emitted nucleic acids. In some embodiments, a fluorescent probe specific to one or several identification sequences is used to facilitate mapping of the reconstructed network with a real image.

An image of the biological sample may be taken by microscopy before step d) or before step e) or between steps d) and e).

The method further comprises dissociating the biological sample and recovering individualized cells or organelles, wherein at least a sub-population of the individualised cells or organelles is labelled with the Emitted nucleic acids that comprise the amplification sequence, the identification sequence, and the capture sequence, or the reverse complement thereof.

Dissociation of the biological sample be achieved using for instance collagenase I, Dnase I and hyaluronidase.

### Method of Mapping and Sequencing Individual Cells or Organelles

The individualized cells labelled with the Emitted nucleic acids as obtainable, or as obtained, by the above method of mapping individual cells or organelles can further be used for sequencing. According to an embodiment, the method of mapping and sequencing individual cells or organelles comprises implementing the method labelling of the invention to provide individualized cells or organelles labelled with Emitted nucleic acids. The individualized cells are thus labelled with (i) a nucleic acid that comprises an amplification sequence, an identification sequence, and a capture sequence, or (ii) the reverse complement thereof.

The method of mapping and sequencing (further) comprises trapping the individualized cells labelled with the Emitted nucleic acids in a compartment, wherein the compartment comprises a compartment-specific nucleic acid and at least one of the following sequences for nucleic acid labeling and further sequencing: hybridization site, ligation site or recombination site.

In an embodiment, single cells or single organelles are trapped in a compartment.

The compartment may comprise a plurality of compartment-specific nucleic acids for targeting specifically different nucleic acids. Preferably, the compartment-specific nucleic acids comprise a barcode specific to the compartment. Preferably, the compartment-specific nucleic acids comprised in the compartment are DNAs.

The compartment-specific nucleic acids may comprise, for example, a 3′-region of sequence oligo d(T) or oligo d(T)VN, for hybridization to poly(A) tail of mRNA (for mRNA sequencing), a 3′-region of sequence complementary to that of a specific RNA (for targeted RNA sequencing) or DNA (for targeted DNA sequencing), or a 3′-region of random sequence, for example d(N)6 (for RNA sequencing or DNA sequencing). More specifically, it is a constant region of a primer containing a compartment-specific sequence that hybridizes to nucleic acids released from the trapped cell(s) or organelle(s).

In an embodiment, the compartment-specific nucleic acids comprise a primer sequence complementary to all or part of the amplification or capture sequence present in the nucleic acid that comprises the amplification sequence, the identification sequence, and the capture sequence, or in the reverse complementary nucleic acid thereof.

As used herein, the term “compartments” denotes for instance droplets, a hydrogel matrix, microfabricated chambers separated by pneumatic valves, microfabricated chambers made with actuatable hydrogels, microfabricated wells, actuatable hydrogel cages or microplate wells.

According to an embodiment, the compartments are wells of a microplate, wherein each well comprise a plurality of oligonucleotides, said oligonucleotides comprising a compartment specific sequence specific to the well, and individualized cells labelled with a nucleic acid comprising an identification sequence are trapped in a well.

According to another embodiment, the compartments are microfabricated chambers made with actuatable hydrogels. In this embodiment of microfabricated chambers made with actuatable hydrogels, the compartments are preferably compartments of a microfluidic device as defined in the section “Microfluidic device” hereafter.

According to an embodiment, the compartments comprise or consists of a hydrogel matrix into which the labelled individualized cells or organelles are embedded to form discrete biological units. According to this embodiment, the labelled individualized cells or organelles are contacted with a plurality of barcode units to form biological unit/barcode unit complexes, and said biological unit/barcode unit complexes are contacted with a hydrogel solution which is then polymerized to embed said biological unit/barcode unit complexes in a hydrogel matrix, wherein each of said biological unit/barcode unit complexes comprises a unique barcode. Hydrogels suitable to form the hydrogel matrix are as described in the international patent application WO2018203141, which is incorporated herein in its entirety. The hydrogel is in particular a thermosensitive or thermoreversible hydrogel, i.e. a hydrogel which, after being formed, depolymerizes if raised above the melting point of the polymer(s) that is contained in the hydrogel.

According to an embodiment, after trapping the individualized cells or organelles, and before subsequent lysing thereof, the method comprises analysing captured cells, organelles and/or molecules they secrete, using optical detection, such as by imaging, including fluorescence imaging, or fluorescence detection.

The method then comprises lysing trapped cells, or cells and organelles, thereby releasing nucleic acids from the cells or organelles in the compartments. Lysis can be performed by any suitable method known to the skilled person. For instance, a solution of low salted water, SDS or Triton X-100, is injected into the compartment to lyse the cells and/or organelles by osmotic shock.

The method further comprises associating i) the compartment-specific nucleic acids with ii) the nucleic acids released from the cell(s) or organelle(s) in the compartments. The association is in particular performed by any one of:
i) hybridizing the compartment-specific nucleic acid by complementarity to the released nucleic acids from the cell(s) or organelle(s);
ii) hybridizing the compartment-specific nucleic acid by complementarity to the released nucleic acids from the cell(s) or organelle(s) and extending the compartment-specific nucleic acid hybridized to the released nucleic acids using a DNA polymerase to create the complementary strand of the released nucleic acids with an associated compartment-specific sequence;
iii) hybridizing the compartment-specific nucleic acid by complementarity to the 3′-end of cDNAs produced by reverse transcription of RNAs from the cell(s) or organelle(s) and extending the cDNA hybridized to the compartment-specific nucleic acid using a DNA polymerase to create the complementary strand of the compartment-specific nucleic acid with an associated compartment-specific sequence;
iv) ligating the compartment-specific nucleic acid to the DNA present in the compartment; or
v) recombining the compartment-specific nucleic acid with the DNA present in the compartment

The compartment-specific sequence and/or nucleic acids released from the cell(s) or organelle(s) are further associated with the Emitted nucleic acids comprising the amplification sequence, the identification sequence, and the capture sequence, or the reverse complement thereof. To that end, the compartment-specific nucleic acids may be designed to comprise a primer sequence complementary to all or part of the amplification or capture sequence present in the Emitted nucleic acid that comprises the amplification sequence, the identification sequence, and the capture sequence, or in the reverse complementary nucleic acid thereof. Then the method additionally comprises hybridizing the compartment-specific nucleic acids to all or part of the amplification or capture sequence present in the Emitted nucleic acid, and extending one or both hybridized DNA strands using a DNA polymerase to create a DNA molecule comprising both the identification sequence, or it's reverse complement, and the compartment-specific sequence, or its complement. Alternatively, instead of the hybridization and extension steps, the method additionally comprises ligating the compartment-specific nucleic acids to all or part of the amplification or capture sequence present in the Emitted nucleic acid, or recombining the compartment-specific nucleic acids to all or part of the amplification or capture sequence present in the Emitted nucleic acid.

The nucleic acids resulting from the association of i) the compartment-specific nucleic acid with ii) the nucleic acids released from the cell(s) or organelle(s) in the compartments and (iii) the Emitted nucleic acids are then recovered and sequenced.

Nucleic acids comprising the same compartment-specific sequence are defined or identified as originating from the same compartment (i.e. from the same single cell or organelle if a single cell or single organelle was trapped into the compartment). The mapping of the position of the single cell or organelle originally in/on the biological sample is based on the identification sequence(s), or reverse complementary sequence thereof, contained in the nucleic acids thus produced, thereby combining mapping and sequencing information of the individual cells of the biological sample. In practice, the mapping of the position of the single cell or organelle (or plurality of cells or organelles contained in a single compartment) originally on the biological sample is performed by determining the relative proportions of identical identification sequence(s), or reverse complementary sequence thereof, contained the nucleic acids produced at step e) to estimate the distance between cells, on the principle of triangulation.

The sequencing information can be mapped back onto an image from microscopy of the biological sample, taken before dissociation.

### Microfluidic Device

In this embodiment, the compartments are compartments of a microfluidic device comprising:
a first wall (12) comprising a first substrate (14) on which a plurality of closed patterns (16) is grafted,
a second wall (18), facing the first wall (12), comprising a second substrate (20),
a plurality of nucleic acids (22) grafted either on the first substrate (14) or on the second substrate (20), each nucleic acid (22) comprising a barcode that encodes the position of the nucleic acid on said first (14) or second (20) substrate,
wherein at least the plurality of closed patterns (16) or the second substrate (20) is made of an actuatable hydrogel which is swellable between a retracted state and a swollen state in which the closed patterns (16) and the second substrate (20) come into contact.

In the swollen state, the closed patterns and the second substrate of the device are in contact. The device thus comprises a plurality of cages, each cage being delimited by a lateral wall made of the closed patterns and by end walls constituted of the first and the second substrates.

In the retracted state, the closed patterns and the second substrate are no more in contact. A gap between the closed patterns and the second substrate allows fluids and cells freely circulating inside the device.

Between the retracted and the swollen states, the device according to the invention goes through a multitude of intermediary states wherein the actuatable hydrogel is only partially swollen. In these configurations, a gap between the closed patterns and the second substrate still exists. However, the height of the gap is sufficiently reduced with respect to the retracted state so that cells, captured in the cages, are retained in the cages. These intermediary configurations may typically be used to allow a selective passage of fluids but not cells.

Each closed pattern thus defines a trapping site for cells wherein closure and opening are initiated by an external stimulus. In a preferred embodiment, the external stimulus is a change in pH, in light intensity, in temperature or in electrical current intensity. In a highly preferred embodiment, the external stimulus is a change in temperature.

The microfluidic device comprises at least 2 closed patterns. Preferably, the microfluidic device comprises a large number of closed patterns, typically 100, 1,000, 10,000, 100,000 . . . .

The first wall and the second wall are made of a rigid material that is capable of resisting temperature fluctuations ranging from −20 to 100° C. According to a first embodiment, the walls (first and/or second walls) are made of a unique and homogenous material. The wall thus consists of the substrates.

According to a second embodiment, the walls further comprise a support material on which is fixed/coated the substrate. Typically, the wall consists of a support material made of glass or polydimethylsiloxane, covered with a substrate layer.

The microfluidic device may equivalently comprise two monolayer walls, two multilayer walls or one monolayer wall and one multilayer wall.

The first substrate is typically made of a material chosen from: silicon, quartz, glass, polydimethylsiloxane, thermoplastics such as cyclic olefin copolymers and polycarbonates, preferably from glass and polydimethylsiloxane. Preferably, the first substrate is made of polydimethylsiloxane.

According to an embodiment, at least part of the surface of the first substrate is structured and/or functionalized.

By “structured”, it is meant that the surface of the substrate is irregular. The surface of the substrate may be porous or microscopically structured. In particular, it can comprise microscopic streaks, pillars, etc . . .

The structuration of the substrate may be performed according to any known process.

Mention may for example be made of standard soft-lithography techniques which are well documented.

By “functionalized”, it is meant that the fixation of chemical functional groups on the surface of the substrate. Typically, the surface of the first substrate is functionalized with chemical groups chosen from hydroxide groups, silanol groups and mixtures thereof, preferably silanol groups.

The structuration and/or the functionalization of the substrate(s) permit to facilitate the grafting of the closed patterns and/or of the nucleic acids on their surface.

According to a preferential embodiment, the first wall is made of a structured and/or functionalized polydimethylsiloxane substrate, preferably of a structured and functionalized polydimethylsiloxane substrate.

The closed patterns may have a large variety of shape. Preferably, the closed patterns are rectangular, square, circular or hexagonal.

Preferably, the closed patterns are covalently grafted to the first substrate.

According to a particular embodiment, the second substrate is made of the hydrogel and the closed patterns are made of a non-swellable material. Preferably, according to this particular embodiment, the second wall comprises a non-swellable support material on which is deposited the swellable hydrogel. The non-swellable support material may be structured and/or functionalized. Structuration and/or functionalization of the non-swellable material are made by analogy with what has been said above in the context of the first substrate. Thus, according to this embodiment, the closed patterns are non-swellable and it is the swelling of the second substrate that permits the closure of the cages.

Preferably, according to this embodiment, the closed patterns are made of a material chosen from silicon, quartz, glass, polydimethylsiloxane, thermoplastic materials such as cyclic olefin copolymers and polycarbonates, preferably from glass or polydimethylsiloxane.

Preferably, the closed patterns have a height ranging from 0.1 to 100 μm, preferably from 1 to 30 μm.

Preferably, the walls of closed patterns have a thickness ranging from 0.1 to 500 μm, preferably from 1 to 20 μm.

Advantageously, according to this embodiment, the second substrate has a thickness, measured in the swollen state into contact with the closed patterns, ranging from 1 to 500 μm, preferably from 1 to 100 μm.

Advantageously, still according to this embodiment, the second substrate comprising the hydrogel has a thickness, measured in the dry state, ranging from 0.5 to 150 μm, preferably from 0.5 to 50 μm.

According to a preferred embodiment, the closed patterns are made of the hydrogel and the second substrate is made of a non-swellable material. Thus, according to this embodiment, the second substrate is non-swellable and the closed patterns swell to close the cages.

Preferably, according to this preferred embodiment, the second substrate is made of a material chosen from: silicon, quartz, glass, polydimethylsiloxane, thermoplastics such as cyclic olefin copolymers and polycarbonates, preferably from glass or polydimethylsiloxane.

Advantageously, the hydrogel patterns have a height, measured in the swollen state when the hydrogel patterns are in contact with the second wall, ranging from 0.1 μm to 500 μm, preferably from 1 μm to 250 μm, more preferably from 1 μm to 100 μm.

Advantageously, the hydrogel pattern has a height, measured in the dry state, ranging from 0.1 μm to 150 μm, preferably from 0.5 μm to 100 μm, more preferably from 0.5 μm to 50 μm.

Preferably, the walls of the hydrogel patterns have a resolution, measured in the swollen state when the hydrogel patterns are in contact with the second wall, ranging from 0.1 μm to 100 μm, preferably from 1 μm to 10 μm.

Preferably, the walls of the hydrogel patterns have a resolution, measured in the dry state, ranging from 0.1 μm to 100 μm, preferably from 0.5 μm to 5 μm.

By “hydrogel”, we refer in the context of a gel comprising a polymer matrix forming a three-dimensional network which is capable of swelling in the presence water, under specific physico-chemical conditions. The swelling of the hydrogel may for example be initiated by a thermal, optical, chemical or electrical stimulus.

For example, the swelling (or the deflation) of the hydrogel may be initiated by a change in temperature, in pressure or in the pH value of the medium wherein it is placed.

Preferably, the hydrogel is a temperature-responsive swellable hydrogel. By “temperature-responsive swellable hydrogel”, we refer in the context of the invention to a hydrogel which swelling or deflation is induced by varying the temperature. A temperature-responsive swellable hydrogel typically exhibits a drastic change of water-solubility with temperature.

In a specific range of temperature, the hydrogel is water-soluble and absorb large quantities of water.

Reversely, by changing the temperature of the medium, the hydrogel becomes no more water-soluble. The hydrogel then releases water and deflates.

By “swollen state”, we refer in the context of the invention to a state of the hydrogel wherein the closed patterns and the second substrate are in contact such that the device comprises a plurality of hermetically sealed cages.

By “retracted state”, we refer in the context of the invention to a state of the hydrogel wherein the closed patterns and the second substrate are not in contact: a gap between the closed patterns and the second substrate exists and permits a free circulation of fluids and cells inside the microfluidic device. The “retracted state” differs from the “dry state” define below in that the hydrogel is not completely free from water. In the retracted state, the hydrogel is still at least partially hydrated.

By “dry state”, we refer in the context of the invention to a state wherein the hydrogel is almost completely free from water. Typically, the hydrogel is in the dry state during the manufacture of the microfluidic device, notably during the grafting of the hydrogel patterns of during the coating of the second wall with the hydrogel substrate.

The temperature at which the water-solubility properties of the hydrogel drastically change is designated as the critical solution temperature (CST).

Preferably, the hydrogel has a critical solution temperature (CST) ranging from 4° C. to 98° C., more preferably from 20° C. to 50° C., even more preferably from 25° C. to 40° C.

According to a first variant, the critical solution temperature (CST) of the hydrogel is a lower critical solution temperature (LCST). At a temperature superior to the LSCT, the hydrogel is in the retracted state and at a temperature inferior to the LCST, the hydrogel is in the swollen state.

According to a second variant, the critical solution temperature (CST) of the hydrogel is an upper critical solution temperature (UCST). At a temperature superior to the UCST, the hydrogel is in the swollen state and at a temperature inferior to the USCT, the hydrogel is in the retracted state.

The polymer constituting the polymer matrix of the hydrogel is typically chosen from homopolymers, copolymers and terpolymers of acrylics, alkyl (meth)acrylates, alkyl (meth)acrylamides, oligoethylene (meth)acrylates, sulfobetaines (meth)acrylates and N-acryloyl glycinamide, preferably chosen from homopolymers copolymers and terpolymers of alkyl (meth)acrylamides and any mixtures thereof, more preferably the hydrogel comprises poly(N-Isopropylacrylamide).

The polymer may be chosen from LCST polymers, UCST polymers and mixtures thereof.

By analogy with what has been said above in the context of the hydrogel:
the expression “LCST polymer” designates a thermo-responsive polymer having a lower critical solution temperature, and
the expression “UCST polymer” designates a thermo-responsive polymer having an upper critical solution temperature.

The overall behavior of the hydrogel (UCST and/or LCST behavior) depends on the nature and on the amount of the different polymers present in the hydrogel.

When the polymer is chosen from UCST polymers, it is preferably chosen from homopolymers, copolymers and terpolymers of acrylics, alkyl (meth)acrylates, alkyl (meth)acrylamides, oligoethylene (meth)acrylates, sulfobetaines (meth)acrylates, N-acryloyl glycinamide and mixtures thereof.

Preferably, the UCST polymer is a terpolymer of methacrylamide, acrylamide and allylmethacrylate.

When the polymer is chosen from LCST polymers, it is preferably chosen from homopolymers, copolymers and terpolymers of acrylics, alkyl (meth)acrylates, alkyl (meth)acrylamides, oligoethylene (meth)acrylates and mixtures thereof, more preferably from homopolymers, copolymers and terpolymers of alkyl (meth)acrylamides, event more preferably the LCST polymer is poly(N-Isopropylacrylamide).

Preferably, the LCST polymer is poly(N-Isopropylacrylamide).

Advantageously, the polymer comprises, preferably consists of, one or several UCST or LCST polymers.

Advantageously, the microfluidic device further comprises at least one inlet (24) and at least one outlet (26) permitting respectively the introduction and the removal of reactants into the device.

Preferably, heating means are integrated in the device according to the invention.

According to an embodiment, each cage comprises independent heating means. This embodiment is particularly advantageous in that it permits to open and close each cage independently.

For example, local heating means may consist of nanoparticles, which heat up when irradiated with light (Plasmonic effect). The nanoparticles may for example be deposited between the hydrogel and the wall on which it is coated or dispersed in the polymer matrix of the hydrogel. Preferably, the nanoparticles are chosen from metal nanoparticles and plasmonic nanoparticles, preferably comprises gold, graphene, silver, copper and titanium nitride.

In another example, local heating is performed using microresistors; for example microresistors comprising chromium/gold bilayer or TiO₂ structures.

The microfluidic device further comprises a plurality of compartment specific nucleic acids grafted either on the first substrate or on the second substrate, wherein each nucleic acid comprises a sequence barcode that encodes the position of the nucleic acid on said first or second substrate.

Advantageously, the nucleic acids are grafted so as to be placed inside the cages, when the hydrogel is in the swollen state.

More advantageously, the nucleic acids are grafted either on the first substrate, inside the closed patterns or on the second substrate, opposite the closed patterns.

Preferably, when the closed patterns are made of the hydrogel, the nucleic acids are grafted on the surface of the second substrate. Preferably, when the second substrate is made of the hydrogel, the nucleic acids are grafted on the surface of the first substrate.

The grafted nucleic acids are RNA or DNA, preferably DNA. The grafted nucleic acids can be single-stranded, double-stranded or partially double-stranded.

The grafted nucleic acids are preferably 60 to 100 nucleotide long.

The grafted nucleic acid may be attached to the substrate at the 3′-end or 5′-end, either directly, or by a linker.

According to an embodiment, grafted nucleic acids sharing the same barcode have a plurality of sequences. According to another embodiment, grafted nucleic acids sharing the same barcode have a same sequence.

According to an embodiment, all or part of the grafted nucleic acids are hybridized to another nucleic acid or a plurality of nucleic acids and form a partly or fully double stranded DNA, double stranded DNA/RNA, or double stranded RNA.

According to an embodiment, the grafted nucleic acids comprise one or any combinations of the following sequences:
1) a restriction site or a photocleavable site for nucleic acid release,
2) a sequence complementary to an amplification primer for further amplification,
3) a T7 RNA polymerase promoter sequence for further in vitro transcription (IVT),
4) a hybridization site for nucleic acid labeling, a ligation site for nucleic acid labeling or a recombination site for nucleic acid labeling, and
5) a sequence of randomized nucleotide residues that function as a unique molecular identifier (UMI).

Preferably, the grafted nucleic acids comprise at least i) a sequence barcode that encodes the position of the nucleic acid on said first or second substrate, and ii) a restriction site or a photocleavable site, and optionally further iii) a primer sequence, and/or T7 sequence and/or hybridization, ligation or recombination site.

According to an embodiment, the grafted nucleic acids of the microfluidic device comprise a constant sequence, i.e. a sequence which is present in all grafted nucleic acids. Grafted nucleic acids of the microfluidic device may be hybridized to a DNA comprising a sequence complementary to all or part of the constant sequence of the grafted nucleic acids. One, or a plurality of different DNA comprising a sequence complementary to all or part of the constant sequence, may be hybridized to the grafted nucleic acids.

The microfluidic device may further comprise structures capable of capturing a cell or an organelle. Such structures are typically chosen from publications as such as Vigneswaran N. et al, 2017, Microfluidic hydrodynamic trapping for single cell analysis: mechanisms, methods and applications, Anal. Methods, 9, 3751-3772.

Preferably, the structures capable of capturing a cell or an organelle are localized either on the first substrate, inside the closed patterns or on the second substrate, opposite the closed patterns Preferably, each cage comprises at least one structure capable of capturing a cell or an organelle.

According to a particular embodiment, a plurality of ligands is grafted, directly or indirectly, covalently or non-covalently, on the first substrate (14) and/or on the second substrate (20), opposite the closed patterns.

Advantageously, the ligands are grafted so as to be placed inside the cages, when the hydrogel is in the swollen state.

In particular, when grafted on the first substrate (14), the ligands are typically grafted inside the closed patterns (16).

Alternatively, when grafted on the second substrate (20), the ligands are facing the closed patterns.

The ligands may all be grafted on the same substrate. Alternatively, some of the ligands are grafted on the first substrate (14) and the others are grafted on the second substrate (20).

Preferably, when grafted directly on the first (14) or second (20) substrate, the plurality of ligands is covalently grafted on the first (14) or second (20) substrate.

According to a more specific embodiment, the plurality of ligands is grafted indirectly: the plurality of ligands is grafted to an intermediate structure, said intermediate structure being directly grafted on the first (14) or second (20) substrate. Thus, according to this specific embodiment, there is no direct bonding between the plurality of ligands and the substrates (14, 20).

Preferably, when grafted indirectly on the first (14) or second (20) substrate, the plurality of ligands is grafted non-covalently on the first (14) or second (20) substrate.

According to first example, the plurality of ligands is conjugated with a nucleic acid and is associated by hybridization to at least part of the grafted nucleic acids (22).

According to another example, the plurality of ligands are non-covalently grafted to an adhesion coating previously coated on the first (14) or second (20) substrate. As adhesion coatings, mentions may notably be made to streptadivin coatings.

In these embodiments, preferably, each ligand is independently chosen from the group consisting of antibodies, fragments of antibody, lectins, and aptamers.

The ligands are usually selected to bind one or more analyte(s) secreted or released by Iysis of the cell(s) or organelles trapped in the cage formed by the first (14) and second (20) walls of the microfluidic device (10), and the closed pattern (16) of hydrogel in swollen state.

Said microfluidic device may be manufactured by a method that comprises the following steps:
1) Providing a first substrate,
2) Grafting on the surface of said first substrate a plurality of closed patterns, 3) Providing a second substrate,
4) Grafting a plurality of nucleic acids either on the surface of the first substrate, or on the surface of the second substrate, wherein each nucleic acid comprises a barcode that encodes the position of the nucleic acid on said first or second substrate;
5) Positioning the first substrate and the second substrate by placing the closed patterns and the nucleic acids between the first substrate and the second substrate,
6) Bonding the first and the second substrates.

The grafting of the closed patterns may be performed according to any known process.

When the closed patterns are made of a non-swellable material, the grafting of the closed patterns is typically performed by soft-lithography techniques.

According to a particular embodiment, the first substrate and the closed patterns are prepared together in a one and unique step.

When the closed patterns are made of the hydrogel, the grafting of the closed pattern is typically performed by photopatterning, preferably under UV (Ultraviolet) radiation. Photopatterning methods consists in the surface-grafting of the polymer matrix of hydrogel on the first substrate, and simultaneously by the crosslinking of the polymer matrix of the hydrogel.

Preferably, the polymers are covalently crosslinked.

More preferably, the crosslinking of the polymer is made in presence of a crosslinking agent chosen from dithiol molecules such as for example dithioerythriol.

The patterning of the hydrogel is typically performed by standard photolithographic techniques or with a direct LASER writing equipment.

These techniques are notably disclosed in Chollet, B., D'Eramo, L., Martwong, E., Li, M., Macron, J., Mai, T. Q., Tabeling, P. and Tran, Y., 2016. Tailoring patterns of surface-attached multiresponsive polymer networks. ACS applied materials & interfaces, 8(37), pp. 24870-24879.

The grafting of the nucleic acids is typically performed by spotting or in-situ light directed synthesis, respectively detailed in DeRisi, J. et al. Use of a cDNA microarray to analyse gene expression. Nat. Genet 14, 457-460 (1996) and in Fodor, S. P. et aL. Light-directed, spatially addressable parallel chemical synthesis. Science (80-.). 251, 767-773 (1991).

Advantageously, during step 5), the first and the second substrates are positioned in a way permitting the nucleic acids to be inside the cages, when the hydrogel is in the swollen state.

More advantageously, the nucleic acids are grafted either on the first substrate, inside the closed patterns or on the second substrate, opposite the closed patterns.

The bonding step may be performed according to any known process.

According to a first embodiment, the bonding step is performed by oxygen plasma treatment. Preferably, the oxygen plasma treatment is made at room temperature, typically at a temperature ranging from 5 to 50° C., more preferably from 10 to 40° C., even more preferably from 15 to 30° C. Preferably, the duration of the oxygen plasma treatment ranges from 10 s to 2 min, more preferably from 30 s to 1 min.

Preferably, according to this first embodiment, the process further comprises, before step 6), a preparation step of deposition on the nucleic acids of a mask capable of protecting the nucleic acids during exposure to the oxygen plasma. The mask is typically made of an adhesive tape, preferably made of a material chosen from plastic film, paper, cloth, foam or foil coated with an adhesive. The mask is finally removed after the plasma treatment, typically by peeling.

According to a second embodiment, the bonding step is performed by the application of a pressure on the surface of the device. Preferably, according to this embodiment, the pressure on the surface of the device is performed by applying a negative pressure into an external microfluidic channel surrounding the main design.

According to third embodiment, the bonding step is performed by using of a crosslinkable composition comprising at least one polymer and optionally at least one crosslinking agent. According to this third embodiment, the bonding step is performed as follows:
a) bringing together the first and the second walls,
b) depositing between the two walls a layer of a composition comprising at least one polymer and at least one crosslinking agent in order to fill the gap between the first wall and the second wall, and
c) crosslinking, preferably self-crosslinking, of the at least one polymer.

Preferably, the polymer is chosen among polyepoxides

The process may further comprise:
between steps 1) and 2), an intermediary step of structuring and/or functionalizing the surface of the first substrate, and/or
between steps 3) and 4), an intermediary step of structuring and/or functionalizing the surface of the second substrate.

When the substrate is made of a hydrogel, the functionalization of the substrate may typically be performed by following the protocol described in Chollet, B. D'eramo, L., Martwong, E., Li, M., Macron, J., Mai, T. Q., Tabeling, P. and Tran, Y., 2016. Tailoring patterns of surface-attached multiresponsive polymer networks. ACS applied materials & interfaces, 8(37), pp. 24870-24879.

If the structures are not made of hydrogel, then functionalization may typically be performed following the protocol detailed in Beal, John H L et al. “A rapid, inexpensive surface treatment for enhanced functionality of polydimethylsiloxane microfluidic channels.” Biomicrofluidics vol. 6,3 36503. 30 Jul. 2012 When the substrate is made of a hydrogel, the structuration of the substrate may typically be performed by following the protocol described in Chollet, B. D'eramo, L., Martwong, E., Li, M., Macron, J., Mai, T. Q., Tabeling, P. and Tran, Y., 2016. Tailoring patterns of surface-attached multiresponsive polymer networks. ACS applied materials & interfaces, 8(37), pp. 24870-24879.

When the substrate is made of a non-swellable material, the structuration of the substrate may typically be performed following standard lithography protocols, notably standard photolithography protocols.

According to a particular embodiment, the process may further comprise, before the deposition the hydrogel material, an additional step consisting of the deposition on the surface of the substrate of a nanoparticle layer, preferably a patterned chromium/gold bilayer.

The deposition of the patterned layer may for example be performed by standard photolithography.

According to a particular embodiment, the method further comprises at least one of the following steps:
a) grafting (directly) a plurality of ligands on the surface of the first substrate (14) and/or on the surface of the second substrate (20), and/or
b) grafting (indirectly) a plurality of ligands on the surface of the first substrate (14) and/or on the surface of the second substrate (20).

Step a) defined above may be performed at any time of the manufacturing method defined above. In particular, step a) may be performed before or after the grafting of the closed patterns (16), before or after the grafting of the nucleic acids (22).

According to a first embodiment, the indirect grafting of ligands is performed by associating to the plurality of grafted nucleic acids (22) a plurality of ligands by hybridization, said plurality of ligands being conjugated with a nucleic acid having complementarity with at least a part of the grafted nucleic acids (22).

According to this first embodiment, step b) is preferably performed after the grafting of the nucleic acids (22). Step b) can be performed until the conditions are modified to actuate the hydrogel into swollen state, thereby trapping cells or organelles in a cage formed by the first (14) and second (20) walls of the microfluidic device (10), and the closed pattern (16) of hydrogel in swollen state.

According to a second embodiment, the indirect grafting of the ligands comprises i) coating an adhesion coating on at least part of the surface of the first (14) and/or second (20) substrate, and ii) grafting the ligands to said adhesion coating.

Step i) can be performed before or after the grafting of the closed patterns (16), before or after the grafting of the nucleic acids (22).

Step ii) is preferably performed after the deposition of the adhesion coating. Step ii) can be performed until the conditions are modified to actuate the hydrogel into swollen state, thereby trapping cells or organelles in a cage formed by the first (14) and second (20) walls of the microfluidic device (10), and the closed pattern (16) of hydrogel in swollen state.

The method for the manufacture of a microfluidic device further comprises one or more of the following steps:
1) Hybridizing a DNA comprising a sequence complementary to all or part of a constant sequence present in grafted nucleic acids, in particular present in all or part of the grafted nucleic acids; in particular, one or a plurality of different DNA comprising a sequence complementary to all or part of the constant sequence may be used;
2) Extending the hybridizing DNA by polymerisation (e.g. using Maxima, SuperScript RT, Phusion or Q5 polymerase);
3) Ligating the grafted nucleic acids, in particular grafted DNA, with a or another DNA sequence; and/or
4) Releasing all or part of the grafted nucleic acid, possibly previously modified by hybridization, extension or ligation according to 1), 2) or 3), from the surface of the first or second substrate, by cleavage (for instance by photocleavage or cleavage catalyzed by an endonuclease).

In some embodiments, the hybridizing DNA forms together with the grafted nucleic acid, a double stranded DNA containing a restriction site for an endonuclease.

The process may also comprise a further step of fixing structures capable of capturing a cell or an organelle.

This supplemental step is typically realized by standard photolithography.

The microfluidic device of the invention can be used in methods of sequencing cells or cell organelles, with the possibility of combining phenotypic information from optical imaging and -omics information for a single cell or organelle, or for e.g. two or more cells in interaction, and this for thousands of cells simultaneously.

The method of performing analysis of cell or organelles comprises:
a) Providing the microfluidic device and a preparation of cells or organelles labelled with Emitted nucleic acids comprising an identification sequence (or reverse complement thereof), as obtainable or obtained by the method of mapping individual cells or organelles of the invention;
b) Optionally, associating all or part of the cells or organelles labelled with the Emitted nucleic acid comprising the identification sequence (or reverse complement thereof) with a common labeling nucleic acid sequence or with a plurality of different labeling nucleic acid sequences;
c) Injecting in the microfluidic device the cells or organelles labelled with the Emitted nucleic acid comprising the identification sequence in suspension under conditions in which the hydrogel is in retracted state;
d) Modifying the conditions to actuate the hydrogel into swollen state, thereby trapping cells or organelles in a cage formed by the first and second walls of the microfluidic device, and the closed pattern of hydrogel in swollen state;
e) Optionally analyzing captured cells or organelles and/or molecules they secrete, using optical imaging;
f) Optionally releasing in the cage, the grafted nucleic acids from the surface of the first or second substrate of the microfluidic device;
g) Optionally, lysing trapped cells or organelles, thereby releasing cellular or organellar nucleic acids in the cages;
h) Associating the barcode of the compartment specific nucleic acids with either the released cellular or organellar nucleic acids and/or Emitted nucleic acid sequence(s) thereby forming barcoded nucleic acids;
i) Modifying the conditions to actuate the hydrogel into the retracted state;
j) Releasing the grafted nucleic acids from the first or second substrate of the microfluidic device, if not released in f);
k) Recovering and sequencing the barcoded nucleic acids; and
l) Optionally mapping the barcoded sequencing data onto the data from optical imaging obtained in e).

According to an embodiment of the method, steps b) and e) are implemented. In some aspects, step b) further comprise labelling of cells with fluorescent markers, that are analysed in step e).

At step c), injecting in the microfluidic device the cells or organelles labelled with the Emitted nucleic acid comprising the identification sequence (or reverse complement thereof) in suspension under conditions in which the hydrogel is in retracted state is typically performed by setting the temperature, pressure or pH—depending on the nature of the actuatable hydrogel—so that the hydrogel is in retracted state. For instance, if the microfluidic device comprises a lower critical solution temperature (LCST) temperature-responsive hydrogel, the temperature of the microfluidic device is raised above the lower critical solution temperature (LCST) to retract the hydrogel. For a temperature-responsive hydrogel comprising or consisting of poly(N-isopropylacrylamide (PNIPAM), the hydrogel is fully expanded at ≤28° C., fully retracted at ≥36° C., and partially expanded between these temperatures, allowing cages to be fully open at 37° C. for cell or organelle loading (D'Eramo et al., Microsystems & Nanoengineering (2018) 4, 17069). For instance, if the microfluidic device comprises an upper critical solution temperature (UCST) temperature-responsive hydrogel, the temperature of the microfluidic device is decreased below the upper critical solution temperature (UCST) to retract the hydrogel. For a temperature-responsive hydrogel comprising or consisting of P(MA-AM-AMA) the hydrogel is fully retracted at ≤10° C., fully expanded at ≥50° C., and partially expanded between these temperatures, allowing cages to be fully open at 10° C. for cell or organelle loading. According to an embodiment, at step d), single cells or single organelles are trapped in the cages. According to another embodiment two (or more) interacting cells are trapped in the cages, for example plasma cell and reporter cell; cytotoxic T cell (or CAR T cell) and target cell (e.g. tumor cell); T cell and antigen-presenting cell.

To actuate the hydrogel into swollen state, the temperature, pressure or pH—depending on the nature of the actuatable hydrogel—is modified so that the hydrogel swells and comes into contact with the second substrate. For instance, if the microfluidic device comprises a lower critical solution temperature (LCST) temperature-responsive hydrogel, the temperature of the microfluidic device is reduced below the lower critical solution temperature (LCST) to swell the hydrogel. For a temperature-responsive hydrogel comprising or consisting of poly(N-isopropylacrylamide) (PNIPAM), the temperature can typically be set at ≤28° C., where the hydrogel is fully expanded (D'Eramo et al., Microsystems & Nanoengineering (2018) 4, 17069). For instance, if the microfluidic device comprises an upper critical solution temperature (UCST) temperature-responsive hydrogel, the temperature of the microfluidic device is raised above the upper critical solution temperature (UCST) to expand the hydrogel. For a temperature-responsive hydrogel comprising or consisting of P(MA-AM-AMA) the hydrogel is fully expanded at ≥50° C.

The method may further comprise, between steps d) and h), changing surrounding conditions of the cells or organelles. Changing surrounding conditions includes circulating in the microfluidic device an aqueous phase containing, e.g. salts, detergents, proteins, and/or nucleic acid sequences. Changing surrounding conditions includes exchanging molecules, such as salts, that pass through the hydrogel of closed cages, by fully opening cages in the case that cages also comprise structures capable of capturing a cell or an organelle, or partially opening the cages.

According to an embodiment, the method further comprises, for example after step e) and before step f), but not necessarily:
e1) Binding of an analyte or of analytes secreted or released by the captured cells or organelles to ligands grafted directly or indirectly to the surface of the first substrate (14) and/or on the surface of the second substrate (20);
e2) Detecting the analyte or analytes bound to the grafted ligand by binding with a labeled second ligand or labeled ligands that is/are specific for the bound analyte or analytes.

According to a first embodiment, at step e2, detecting is performed directly with a second ligand or ligands fluorescently labeled.

According to a second embodiment, at step e2, detecting is performed indirectly, with a second ligand or ligands labeled with a ligand identification nucleic acid to the analyte or analytes bound to the grafted ligand, wherein the sequence of said ligand identification nucleic acid allows identification of the ligand and the analyte or analytes bound to the grafted ligand.

According to this second embodiment, the method may further comprise amplifying the sequence of said ligand identification nucleic acid. Amplification preferably consists of a linear amplification, more preferably by using at least one polymerase and at least one restriction or nicking enzyme.

According to this second embodiment of the method, in step h), the method may further comprise associating the barcode of the nucleic acids (22) with the ligand identification nucleic acid, thereby forming barcoded nucleic acids.

According to an embodiment, the common labeling DNA sequence or plurality of different labeling DNA sequences provided at step b) are used for DNA-toolbox reactions (or dynamic DNA reaction network) for phenotype sorting of cells or organelles, thereby actuating the release of the grafted nucleic acids in step f) or j). The principles of DNA-toolbox reactions are described for instance in the international patent applications WO2017141068 and WO2017141067.

According to an embodiment, at step g), trapped cells or organelles are lysed by osmotic shock. This may be readily implemented by the skilled person by circulating in the microfluidic device a hypo- or hyper-osmotic aqueous phase. The cages may be retained closed for this operation.

According to an embodiment, step h) comprises hybridizing the compartment-specific nucleic acids comprising barcodes, which may be still grafted the surface of the first or second substrate of the microfluidic device or released from the surface of the first or second substrate of the microfluidic device, by complementarity to the released cellular or organellar nucleic acids and/or to Emitted nucleic acid sequence(s). In particular, where the compartment-specific nucleic acids comprising a barcode are DNA, step h) [or the method between steps i) and j)] may additionally comprise extending the DNA comprising a barcode hybridized to the released cellular or organellar nucleic acids (or labeling Emitted nucleic acid sequence(s)) using a DNA polymerase to create the complementary strand of the released cellular or organellar nucleic acids (or labeling nucleic acid sequence(s)) which comprises a barcode. The nucleic acids may comprise, for example, a 3′-region of sequence oligo d(T) or oligo d(T)VN, for hybridization to the poly(A) tail of mRNA (for mRNA sequencing), a 3′-region of sequence complementary to that of a specific RNA (for targeted RNA sequencing) or DNA (for targeted DNA sequencing), a 3′-region of random sequence, for example d(N)6 (for RNA sequencing or DNA sequencing), a 3′-region with three ribo(G) nucleotides for reverse transcriptase template switching (for RNA sequencing), or a 3′-region complementary to a nucleotide sequence introduced by recombination, for example after “tagmentation” catalyzed by Tn5 transposase. The latter can be used, for example, for genomic DNA sequencing, or epigenomic analysis of DNA methylation (using Methyl-seq or bisulfite sequencing) or chromatin structure (using transposase-accessible chromatin with sequencing, ATAC-Seq), or for RNA sequencing after tagmentation of RNA-DNA duplexes formed after first strand cDNA synthesis or double-stranded DNA formed after first and second strand cDNA synthesis on RNA released by the cells or organelles.

According to another embodiment, the compartment-specific nucleic acids comprising a barcode are DNA, and may be wholly or partly double-stranded, and step h) comprises ligating the DNA comprising a barcode to DNA released by the cells or organelles. For example, the barcodes may be ligated to genomic DNA, for example after restriction digestion (for genomic DNA sequencing or analysis of DNA methylation), or after digestion with micrococcal nuclease (for metagenomic analysis using MNase-seq or ChIP-seq).

According to still another embodiment, the compartment-specific nucleic acids comprising a barcode are DNA, and may be wholly or partly double-stranded, and step h) comprises recombining the DNA comprising barcode with DNA released by the cells or organelles. For example, the barcodes may be recombined with genomic DNA for genomic DNA sequencing, or epigenomic analysis of DNA methylation (using Methyl-seq or bisulfite sequencing) or chromatin structure (using transposase-accessible chromatin with sequencing, ATAC-Seq). Alternatively, the nucleic acids comprising a barcode recombine with RNA-DNA duplexes formed after first strand cDNA synthesis on RNA released by the cells or organelles, or recombining with double-stranded DNA formed after first and second strand cDNA synthesis on RNA released by the cells or organelles (for RNA sequencing). In a preferred embodiment the oligonucleotide comprises a Mosaic End (ME) sequence which recombines with DNA catalyzed by Tn5 transposase.

According to an embodiment, the method further comprises between steps d) and h), releasing the compartment-specific nucleic acids comprising barcodes upon the presence of a cellular or organellar material (e.g. a surface molecule, a secreted molecule, or a lysis product) in the cages, e.g. by a proximity ligation assay, or proximity extension assay.

### Kits for Mapping and Sequencing Individual Cells or Organelles

The invention further relates to a kit for implementing the above method of mapping and sequencing which comprises the constituent of the kit for labelling individual cells or organelles as defined above and a compartment as defined above.

The kit comprises:
a) Emitter nucleic acids wherein each emitter nucleic acid comprises an amplification sequence, an identification sequence, and a capture sequence;
b) Receptor nucleic acids wherein each receptor nucleic acid comprises i) an amplification sequence matching all or part of the amplification sequence of the set of Emitter nucleic acids, or a complement thereof, or ii) a capture sequence matching all or part of the capture sequence of the set of Emitter nucleic acids, or a complement thereof;
c) Ligands of a cell target or organelle target;
d) Optionally a nicking endonuclease and a polymerase with strand displacement activity.

In some embodiments, the set of Emitter nucleic acids is provided in the form of group(s) of Emitter nucleic acids wherein all Emitter nucleic acids of a group comprise an identical identification sequence. In this embodiment the kit further comprises an endonuclease.

The invention will be further illustrated in view of the following figures and examples.

### FIGURES

FIG. 1: Flow cytometry fluorescent measurement of unstained tissue (negative control), tissue stained before tissue dissociation (pre-dissociation staining) and tissue stained after tissue dissociation (post-dissociation staining). Bottom figures correspond to a labeling with ubiquitous and fluorescent cell surface markers, including antibody anti-human CD98 and lectin PHA. Top figures correspond to a labeling with ubiquitous cell surface markers carrying a fluorescent and biotinylated oligonucleotide conjugated via a streptavidin (ASO: Antibody Streptavidin Oligonucleotide). A separation is observed between stained fluorescent and non-stained cells, indicating a resistance of the staining to the dissociation.

FIG. 2: The combination of enzymes and oligonucleotides present in the solution can be used to perform local amplification of barcoded nucleic acids, for example, under the action of a polymerase with strand displacement activity and a nicking endonuclease. A is an amplification sequence, B is an identification sequence, C is a capture sequence. A and B are needed for later single-cell analysis and library preparation. N a priming sequence containing a recognition site at its 5′ for a nicking endonuclease. A, B, C, N are respectively complementary nucleic acids of A, B, C, N. Four different embodiments are shown, wherein the oligonucleotides, called Emitter nucleic acids, are captured onto the cell surface via an antibody (“r”) specific to a cell-surface marker, however, other ligands binding the cell membrane can also be used in place of the antibody.
1. The 3′ part of the antibody-conjugated oligonucleotide (antibody “e”), called Emitter, is directly self-primed for polymerization. After polymerization, the nicking endonuclease can create a nick allowing the strand-displacing polymerase to prime and polymerise again while displacing the strand complementary to the emitter oligonucleotide. Released nucleic acids can diffuse and hybridise to available and complementary antibody-conjugated receptor oligonucleotides (“r”).
2. The 3′ part of the antibody-conjugated oligonucleotide (antibody “e”), called Emitter, is primed by free oligonucleotide (N C̅) for nicking and polymerization. The nicking endonuclease can create a nick allowing the strand-displacing polymerase to prime and polymerise again while releasing the complementary nucleic acid of emitter oligonucleotide. Released nucleic acids can diffuse and hybridise to available and complementary antibody-conjugated receptor oligonucleotides (“r”). After polymerization, the nicking endonuclease can create a nick allowing the strand-displacing polymerase to prime and polymerise again while releasing a new emitter oligonucleotide.
3. The 3′ part of the antibody-conjugated oligonucleotide (antibody “e”), called Emitter, is directly self-primed for polymerisation. After polymerisation, the nicking endonuclease can create a nick allowing the strand-displacing polymerase to prime and polymerise again while releasing the complementary strand of emitter oligonucleotide. Released nucleic acids can hybridise to free partially complementary oligonucleotide, allowing a new polymerisation and amplification cycle. Released nucleic acids can diffuse and hybridise to free antibody-conjugated receptor oligonucleotide (“r”).
4. The 3′ part of the antibody-conjugated oligonucleotide (antibody “e”), called Emitter, is directly self-primed for nicking and polymerization. The nicking endonuclease can create a nick allowing the strand-displacing polymerase to prime and polymerise again while releasing the complementary strand of emitter oligonucleotide. N′ corresponds to the same sequence as N where at least one thymine from the recognition site has been replaced by a uracil to avoid recognition and nicking by the nicking endonuclease. Though after polymerisation, the strand complementary to N′ will be N̅. Released nucleic acids can directly self-hybridise by forming a hairpin structure and allowing a second polymerisation and nicking cycle.

Each hairpin structure is composed of different nucleic acids sequences to avoid unwanted folding. Released nucleic acids from the second polymerisation and nicking cycle can diffuse and hybridise to free antibody-conjugated receptor oligonucleotide (“r”).

FIG. 3: a. Measurement of double stranded DNA production starting from 10 nM, 1 nM, 100 pM, 10 pM, 1 pM, 0.1 pM and 0 pM of emitter. Reaction mixtures are made according to Table 2. 90 min incubation at 37° C. and fluorescent measurement were done using a thermocycler (CFX384 Touch, Biorad) with 2 cycles per minutes. b. Amplification strategy used for the isothermal production of oligonucleotides presented in FIG. 2.1. A is an amplification sequence, B is an identification sequence, C is a capture sequence. A and B are needed for later single-cell analysis and library preparation. N a recognition site for a nicking endonuclease. Q is a sequence is to hybridize a quencher, represented as a black sphere. S is a spacer to move away the biotin, represented as a square. Ā, B̅, C̅, N̅ and Q̅ are respectively complementary nucleic acids of A, B, C, N and Q. In step 1, the self-primed emitter (SEQ ID NO:2) releases nucleic acids, represented in step 2. In step 3, the released nucleic acids hybridized to a fluorescent reporter (“QAS”, SEQ ID NO:4). The reporter is partially hybridized to a sequence carrying a quencher (“Q̅”, SEQ ID NO:5), preventing the observation of fluorescence, In step 4, the sequence carrying a quencher is released by polymerization of the released nucleic acids on the reporter, causing the appearance of the fluorescent signal. c. Electrophoresis of amplification products and standards. 5 μl per line, 4% agarose gel (Thermo Scientific R2802). Migration at 120 V for 30 min in 1× TAE (Dutscher 348605). 3 μl of FastRuler Low Range DNA Ladder (Thermo Scientific SM1233) per ladder line. Concentration standards made with serial dilutions of double stranded ABC oligonucleotides, from 1 μM to 10 nM in same conditions as described for amplifications products.

FIGS. 4-5: Co-culture on a 48-well plate of HUVEC and HEPG2 cells. Each thumbnail represents the same observation field but in different observation channels and at two different time: T0 and T1 (40 min after T0). A. brightfield image showing the co-culture of the two cell lines. B. A1647 fluorescent channel (1 s exposition), showing anti-human CD146 antibody labeling, specific to the HUVEC cells. C. BV421 fluorescent channel (1 s exposition), showing anti-human CD326 antibody labeling, specific to the HEPG2 cells. The emitter sequence (SEQ ID NO:2) is also conjugated with anti-human CD326 antibody. D. ATT0488 fluorescent channel (1 s exposition), showing the reporter signal, conjugated with anti-human CD146 antibody and therefore present at the surface of HUVEC cells. At T0, the co-culture is incubated at 37° C. with the reaction mix detailed in Table 3, allowing the self-primed emitter (SEQ ID NO:2) carried by the HEPG2 cells to release partially random nucleic acids. The fluorescent reporter (SEQ ID NO:9) present at the surface of the HUVEC cells is partially hybridized to a sequence carrying a quencher (SEQ ID NO:5), preventing the observation of fluorescence at TO. After diffusion and amplification, the released nucleic acids hybridized to the fluorescent reporter (SEQ ID NO:9). At T1, the sequence carrying a quencher is released by polymerization of the released nucleic acids on the reporter, causing the appearance of the fluorescent signal. The white arrow points a HUVEC cell, as shown by the AI647 fluorescent channel. At T0 there is no signal in the ATT0488 channel, indicating that all the fluorescent reporters (SEQ ID NO:9) on that cell are partially hybridized with a sequence carrying a quencher (ID4). At T1, there is a signal in the ATT0488 channel, colocalized with the staining in A1647 channel on that cell, indicating that the sequence carrying a quencher (SEQ ID NO:3) has been displaced, as a consequence of the release, amplification and diffusion of nucleic acids from the self-primed emitter (SEQ ID NO:2).

FIG. 6: schematic representation of the closed patterns of a microfluidic device usable according to the invention. The microfluidic device 10 comprises a plurality of closed patterns 16 arranged in the form of a table. The closed patterns 16 represented are in the form of squares but may equivalently in the form of rectangles, circles or even hexagons. The closed patterns 16 have a thickness e and a height h.

FIG. 7: schematic representation of a microfluidic device wherein the patterns are made of an actuatable hydrogel. The microfluidic device 10 comprises a first wall 12 comprising a first substrate 14 on which a plurality of closed patterns 16 is grafted. A second wall 18, facing the first wall 12, comprises a second substrate 20. A plurality of nucleic acids 22 are grafted on the second substrate 20. The closed patterns 16 are made of an actuatable hydrogel which is swellable between a retracted state and a swollen state in which the closed patterns 16 and the second substrate 20 come into contact. The microfluidic device 10 further comprises an inlet 24 and an outlet 26 permitting respectively the introduction and the removal of reactants into the device 10.

On scheme A, the closed patterns 16 are in the retracted state. A gap 28 between the closed patterns 14 and the second substrate 20 allows fluids and cells present inside the device freely circulating inside the device 10.

Placed in specific physico-chemical conditions, the closed patterns 16 begin to absorb water and swell. The closed patterns 16 thus elongates until contacting the second substrate 20.

On scheme B, the closed patterns 16 are in the swollen state, in contact with the second substrate 20. The device 10 thus comprises a plurality of cages 30, each cage 30 being delimited by a lateral wall made of one of the closed patterns 16 and by end walls constituted of a portion of the first 14 and second 20 substrates.

FIG. 8: schematic representation of a microfluidic device wherein the second substrate is made of an actuatable hydrogel. The microfluidic device 10 comprises a first wall 12 comprising a first substrate 14 on which a plurality of closed patterns 16 is grafted. A second wall 18, facing the first wall 12, comprises a second substrate 20. A plurality of nucleic acids 22 are grafted on the first substrate 14. The second substrate 20 is made of an actuatable hydrogel which is swellable between a retraced state and a swollen state in which the closed patterns 16 and the second substrate 20 come into contact. The microfluidic device 10 further comprises an inlet 24 and an outlet 26 permitting respectively the introduction and the removal of reactants into the device 10.

On scheme A, the second substrate 20 is in the retraced state. A gap 28 between the closed patterns 14 and the second substrate 20 allows fluids and cells present inside the device freely circulating inside the device 10.

Placed in specific physico-chemical conditions, the second substrate 20 begins to absorb water and swells. The thickness of the second substrate 20 thus increases until contacting the closed patterns 16.

On scheme B, the second substrate 20 is in the swollen state, in contact with the closed patterns 16. The device 10 thus comprises a plurality of cages 30, each cage 30 being delimited by a lateral wall made of one of the closed patterns 16 and by end walls constituted of a portion of the first 14 and second 20 substrates.

### EXAMPLES

### Example 1: Preserved DNA Coupling with Cells During Dissociation

To achieve a proof of concept applicable to a cohort of biological samples, we demonstrated that we could preserve the coupling of a DNA nucleic acids with cells, using antibodies, lectins or cholesterol-tag as ligand, from a tissue section during dissociation independently of the cell type.

### Cell Culture

Jurkat human T lymphocyte ATCC® TIB-152 and Ramos human B lymphocyte ATCC® CRL-1923 are cultivated in RPMI 1640 Medium (Gibco 61870044) supplemented with 10% heat inactivated Fetal Bovine Serum (Gibco 10082147) and 1% Penicillin-Streptomycin (Gibco 15140122). Cells are seeded on 25 cm2 or 75 cm2 culture flask at 37° C. with 5% CO2 following ATCC recommendations. On reaching 75-80% confluence the cells are diluted. After retrieving from cell culture, the cells are finally re-suspended in TBS 1× at the concentration of 2.10⁶ cells.mL⁻¹.

### Antibody and Lectin Conjugation

Purified antibody (Biolegend) and lectin (Eurobio Scientific) are firstly conjugated with streptavidin using Streptavidin Conjugation Kit Protocol (ab102921). Conjugated markers are then mixed in 1× tris buffered saline (TBS, VWR CAYM600232-500) with biotinylated oligonucleotides in 1:12 ratio and stored protected from light in a room with controlled temperature between 2° and 25° C. for over 12 h (over-night). Biotinylated oligonucleotides are purchased from IDT, in a 100 μM concentration in IDTE Buffer, pH 8.0, with standard desalting. The sequence of the biotinylated fluorescent oligonucleotide is: /56-FAM/CACAGGGTGATCAGGT/3Bio/(SEQ ID NO:1). 56-FAM stands for a fluorescein fluorescent dye attached at the 5′ end of the oligo, 3Bio for a biotin attached at the 3′ end of the oligo.

### Tissue Dissociation

About 2 g of fresh colon sample were cut in pieces of about a mm². Before proceeding to staining, pieces of tissue were washed three times in 10 ml of 1× phosphate buffered saline (PBS, Gibco 10010023) followed by three washes in Cell Staining Buffer (Biolegend 420201) with DSS (Sigma D8906) at 400 μg/ml and 5 mM EDTA (Sigma 03690). Tissue was stained with 1 to 10 μg of antibodies or lectins conjugated with a fluorophore or a fluorescent oligonucleotide, in 500 μl of Cell Staining Buffer (Biolegend 420201) for 30 min at 4° C. Pieces were then washed in 10 ml of 1× phosphate buffered saline before proceeding to dissociation using gentleMACS Octo Dissociator and Tumor Dissociation Kit (Miltenyi Biotec 130-095-929). After dissociation cells were filtered at 40 μm, washed 10 ml of tris buffered saline (TBS, VWR CAYM600232-500) and resuspended in 1 ml TBS. Optionally, cells were stained with DAPI to distinguish living and dead cells.

### Cell Staining

200,000 cells are resuspended in 100 μL of Cell Staining Buffer (Biolegend 420201) with DSS (Sigma D8906) at 400 μg/ml and 5 mM EDTA (Sigma 03690). Cells were incubated with 5 μL of Fc Receptor Blocking Solution (Biolegend 422301) in the dark at 4° C. for 10 min followed by the addition of 0.2 to 2 μg of antibodies or lectins conjugated with a fluorophore or fluorescent oligonucleotides or equivalent quantity of cholesterol modified oligonucleotides. Cells were incubated for 30 min in the dark at 4° C. before being rinse twice in the Cell Staining Buffer mix previously described and twice in Tris Buffered Saline (TBS, VWR CAYM600232-500). For each wash, cells were centrifugated for 5 min at 130 rcf and 4° C., supernatant was removed except 50 μl before 200 μl of clean buffer was added. After the last wash, cells were resuspended in 200 μl TBS.

Cell staining is usually performed after tissue dissociation, however in order to labeled cells according to their initial position in the tissue, the staining needs to be done prior to dissociation.

We selected universal external cell markers in order to label every cell of the tissue, without need of permeabilization.

We firstly demonstrated using flow cytometry (Guava easyCyte 12HT) markers un-specificity and absence of marker exchange after staining on Jurkat and Ramos cell lines. Universal cell markers were chosen among anti-human CD98 (BioLegend 315603, 315602), anti-human CD298 (BioLegend 341709) or anti-human 32-microglobulin (BioLegend 316317, 316302), lectin jacalin, lectin LCA, lectin PHA-E and a cholesterol modification (3CholTeg at IDT with HPLC purification) at the 3′ end of the oligo in place of the biotin modification.

We stained each population with one of the markers before mixing a part of each stained population together for 30 min and analyzing them through flow cytometry. It appears that we were still able to distinguish each population after mixing, whatever the type of labeling, indicating an absence of cross-contamination after staining (data not shown). When using the oligo with the cholesterol modification, the separation is less important than for the antibodies or lectins but still present,

We also demonstrated that antibody conjugated oligonucleotide do not exchange their oligonucleotide via the biotin streptavidin linkage by mixing a population stained with markers conjugated with fluorescent oligonucleotides and a population stained with markers conjugated with non-fluorescent oligonucleotides (data not shown).

Finally, we demonstrated the resistance of selected cell markers to tissue dissociation. Each time, we compared unstained tissue with tissue stained before and after dissociation, and we assessed the presence of the staining by flow cytometry (Guava easyCyte 12HT).

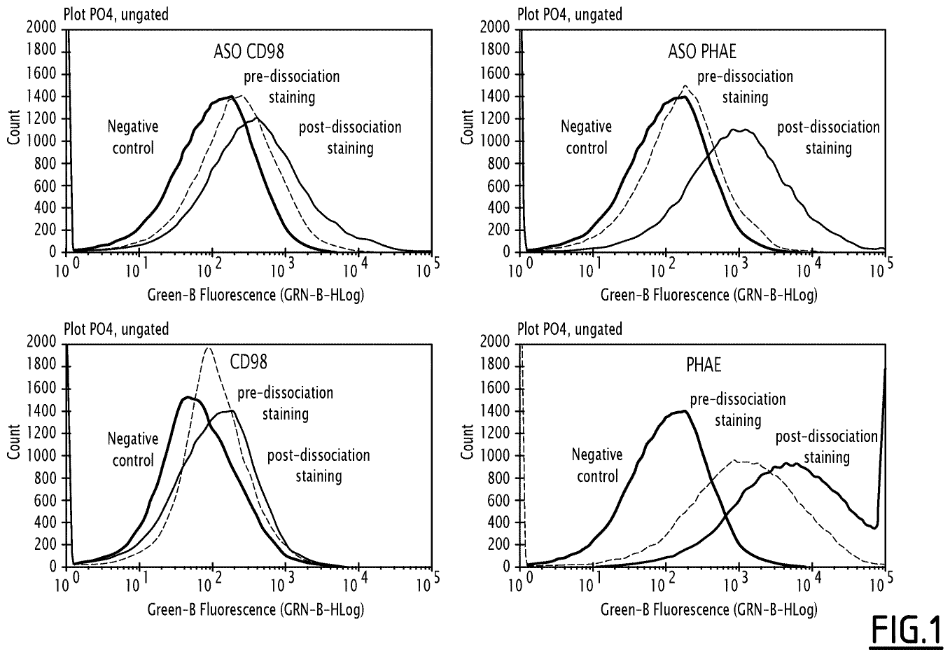

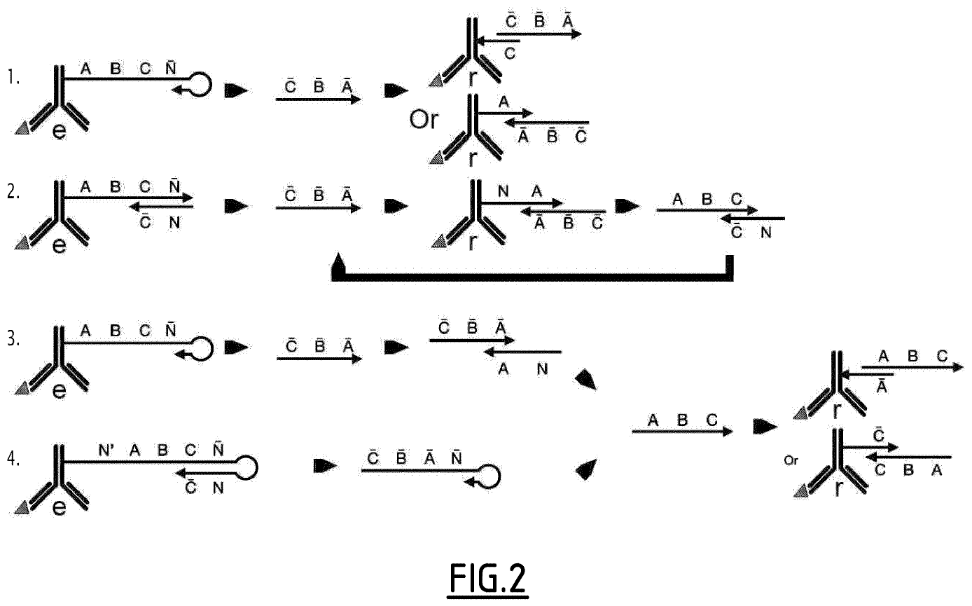

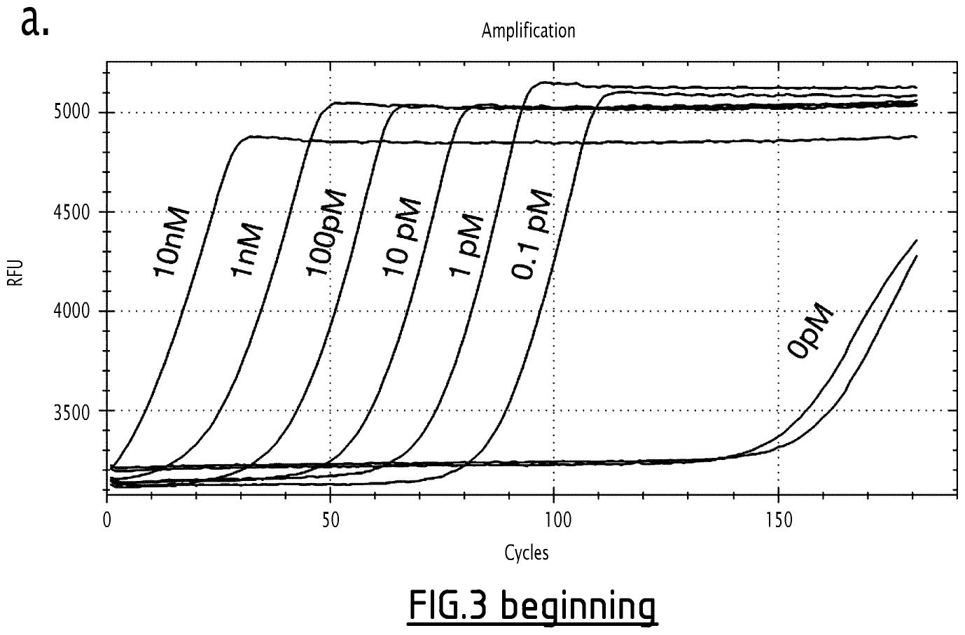

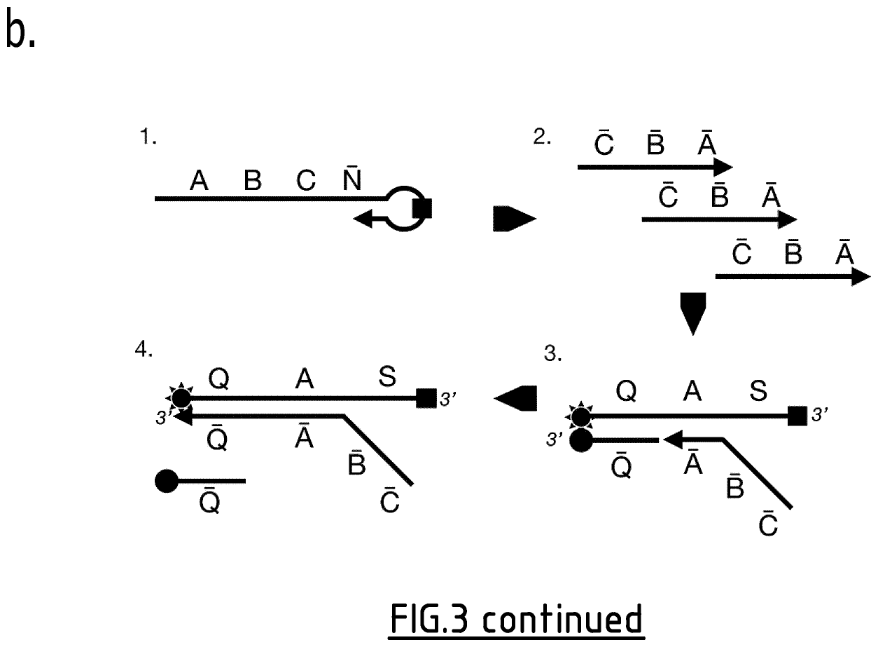

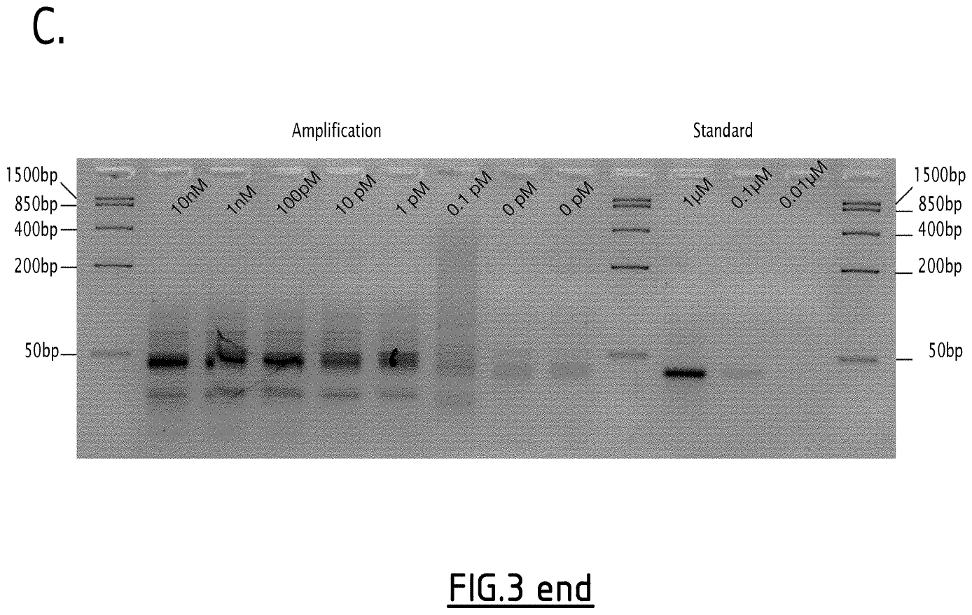

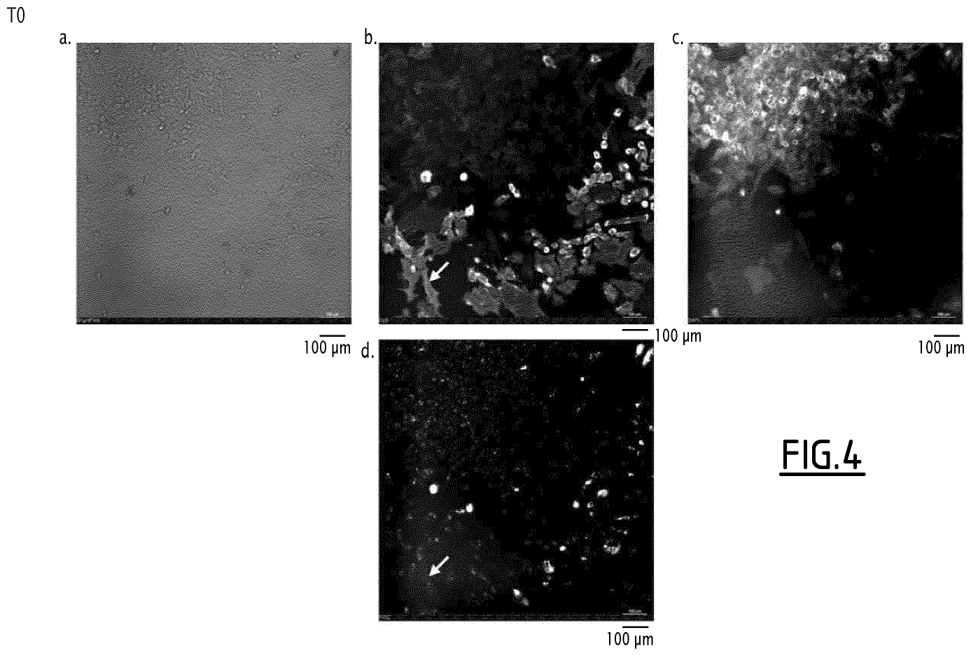

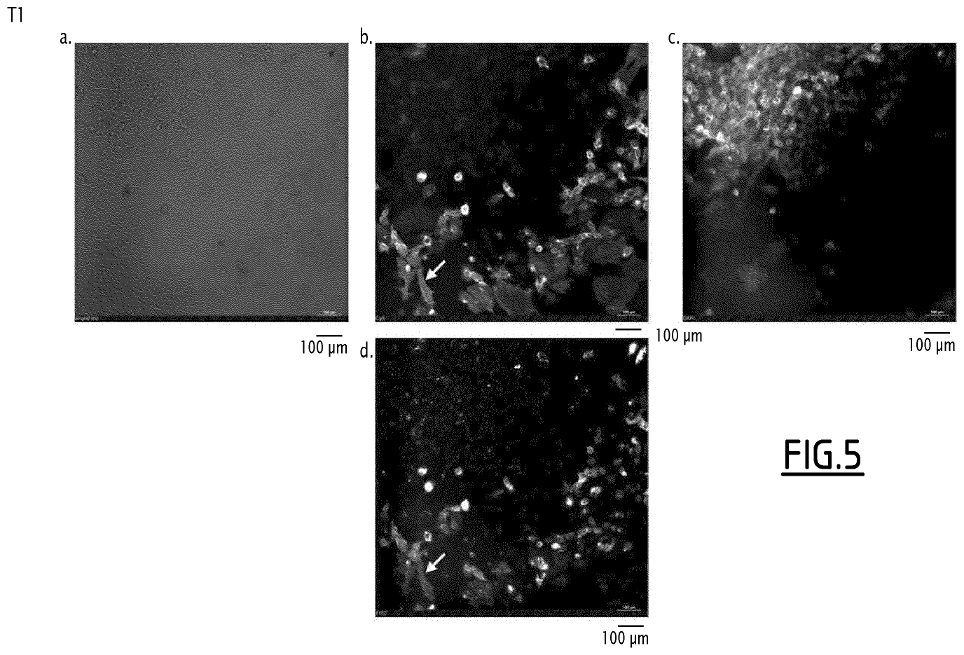

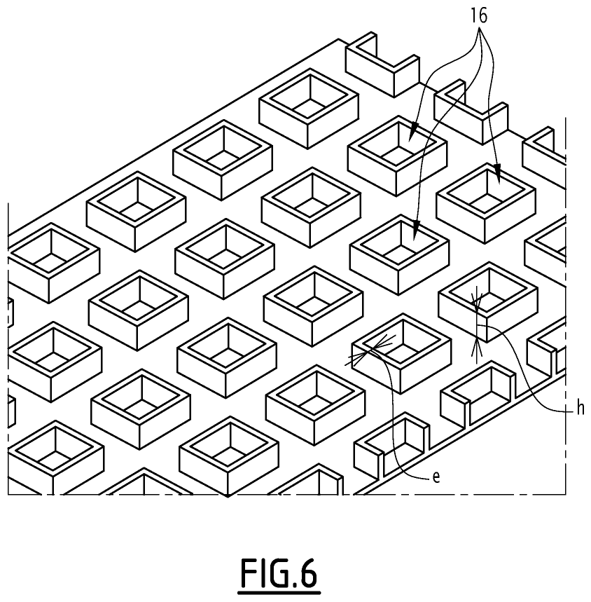

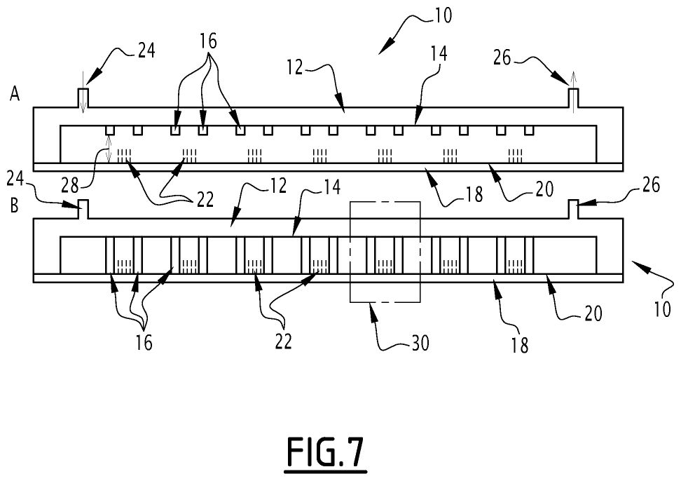

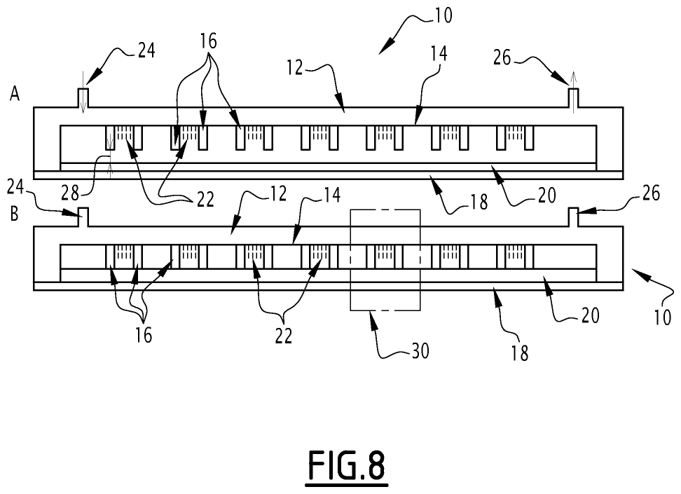

With antibodies or lectins, using a fluorophore or a conjugation with a fluorescent oligonucleotide, the labeling was partially kept during the tissue dissociation (FIG. 1). The difference of staining between the pre- and post-dissociation conditions may be explained by the inability of markers to fully stain the inner part of tissue blocks.

In parallel, we confirmed that we could recover both transcriptomic and labeling nucleic acid information associated with the same cell, with a method similar to Cite-seq method (Stoeckius et al., Nat Methods. 2017 September; 14(9):865-868).

### Example 2: Isothermal Amplifications of Emitter Nucleic Acids

We also tested several isothermal amplifications with the goal to get more than a thousand-fold amplification of the oligonucleotides, using similar order of magnitude of reagents and nucleic acids that will be used on a tissue. Examples of procedure for such amplifications are described in FIG. 2.

### Oligonucleotides

Oligonucleotides were design in silico and checked to limit unwanted hairpin, homodimer or heterodimer structures using RNAstructure and PrimerBlast. Oligonucleotides were purchased from IDT, at 100 μM concentration in IDTE Buffer, pH 8.0, with standard desalting. /iBiodT/modification stands for internal biotin, /56-FAM/modification stands for 5′ fluorescein, /3Bio/modification stands for 3′ biotin, /3IABkFQ/modifications stands for 3′ quencher with absorbance spectra ranging from 420 to 620 nm with peak absorbance at 531 nm, and/5ATTO488N/modification stands for 5′ ATTO 488 fluorophore.

TABLE 1
example of oligonucleotide design for isothermal amplification and reporting

| Sequence | Function |
|---|---|
| AACACCAAACCCTTCTAAAGCCCAAACCTCNNNNNNNNNNNNA | Emitter with |
| AGCGATCTGTTACCAAGCCGTCCTCAGCAGGATTAGAGTT/iBiod | 12 bp random |
| T/TTCTCTAATCCTGC (SEQ ID NO: 2) | sequence |
| AACACCAAACCCTTCTAAAGCCCAAACCTC (SEQ ID NO: 3) | Receptor |
| /56-FAM/ACACGTTGATCTAGTCGCCACCAACACCAAACCCTTCT | Reporter |
| AAAGCCCAAACCTCCATTCGTTCCGCTCGCAACAAT/3Bio/ | |
| (SEQ ID NO: 4) | |
| GGTGGCGACTAGATCAACGTGT/3IABkFQ/ (SEQ ID NO: 5) | Quencher |
| ACGGCTTGGTAACAGAT (SEQ ID NO: 6) | Sequencing |
| | primer 1 |
| AACACCAAACCCTTCTAAAG (SEQ ID NO: 7) | Sequencing |
| | primer 2 |
| ACACGTTGATCTAGTCGCCACC (SEQ ID NO: 8) | Sequencing |
| | primer 3 |
| /5ATTO488N/ACACGTTGATCTAGTCGCCACCAACACCAAACCCT | Reporter 2 |
| TCTAAAGCCCAAACCTC/3Bio/ (SEQ ID NO: 9) | |
| AACACCAAACCCTTCTAAAGCCCAAACCTC/3Bio/ | Biotinylated |
| (SEQ ID NO: 10) | receptor |
| ACGGCTTGGTAACAGATCGCTT (SEQ ID NO: 11) | Amplification |
| | sequence |

### Reaction Mixture Assembly

All reactions were incubated at 37° C. for 90 min in a thermocycler (CFX384 Touch, Biorad), with a measurement of SYBR fluorescence every 30 seconds. Reaction mixture was made according to Table 2.

TABLE 2
Reaction mixture.

| | Final | Concentration | |
|---|---|---|---|
| | Concentration | unit | in μL |
| DNase/RNase-Free | | | 5.90 |
| Distilled Water | | | |
| (Invitrogen 10977023) | | | |
| Emitter (SEQ ID NO: 2) | 10⁻² to 10⁻⁷ | μM | 1.00 |
| Receptor (SEQ ID NO: 3) | 0.1 | μM | 1.00 |
| Reporter (SEQ ID NO: 4) | 0.1 | μM | 0.33 |
| Quencher (SEQ ID NO: 5) | 0.3 | μM | 0.66 |
| Bst DNA Polymerase, | 0.2 | U/μl | 0.20 |
| Large Fragment | | | |
| (NEB M0275S) | | | |
| Nb.BbvCI (NEB R0631S) | 0.2 | U/μl | 0.20 |
| NEB 2 Buffer (B7002S) | 1 | x | 1.00 |
| dNTPs (NEB N0447L) | 0.2 | mM | 0.20 |
| Final Reaction Volume | | | 10.00 |

In these conditions, we found that the Emitter oligonucleotide can produce more than a million sequences starting from concentration down to 0.1 μM (FIG. 3).

The consistency of the amplification has been confirmed by Sanger sequencing (LightRun form Eurofins, using primers (SEQ ID NO:6, SEQ ID NO:7 and SEQ ID NO:8) after DNA extraction from an agarose gel electrophoresis (similar to the one shown in FIG. 3c).

The inventors have also tested other polymerases (bst, bsu, phi29), nicking endonucleases (Nt.Alwl, Nb.Btsl) and using a different buffer (NEB Cutsmart). The inventors have also designed and tested other oligonucleotide designs based on the same amplification strategy and also with different amplification strategies (as described in FIG. 2). The results show that these embodiments can also be performed correctly.

### Example 3: Isothermal Amplifications of Emitter Nucleic Acids and Capture on Receptor Nucleic Acids Based on 2D Layer of Living Cells

The inventors have then demonstrated the ability to generate and capture random barcode directly on a 2D layer of living cells.

### Cell Culture

HEPG2 (ATCC HB-8065) are cultivated in DMEM (41965039), with 10% heat inactivated Fetal Bovine Serum (Gibco 10082147) and 1% Penicillin-Streptomycin (Gibco 15140122). Cells are seeded on 25 cm2 or 75 cm2 culture flask at 37° C. with 5% CO2 following ATCC recommendations. On reaching 75-80% confluence the cells are subcultured.

HUVEC (Lonza C2519A) cells are cultivated in Endothelial Cell Growth Medium (Promocel C-22010) and 1% Penicillin-Streptomycin (Gibco 15140122). Cells are seeded on 12.5 cm2 or 25 cm2 culture flask at 37° C. with 5% CO₂ following Lonza recommendations. Every 48 hours, half of the medium is replaced and on reaching 75-80% confluence the cells are subcultured.

HUVEC are then subculture in 48-well plate in 250 μl of from the aforementioned medium. After 48 hours, half of the medium is replaced and HEPG2 are added 0.3% of a 25 cm2 culture per well. 48 to 72 hours later, the co-culture is ready to be stained.

### Antibody Conjugation

Purified anti-human CD146 (Biolegend 361002) and Purified anti-human CD326 (Biolegend 324202) are conjugated with streptavidin using Streptavidin Conjugation Kit (Abcam 102921). Conjugated antibodies are then mixed in 1× tris buffered saline (TBS, VWR CAYM600232-500) with biotinylated oligonucleotides in 1:12 ratio and stored protected from light in a room with controlled temperature between 2° and 25° C. for over 12 h (over-night). Purified anti-human CD146 is conjugated with the sequence consisting of SED ID NO: 8 and Purified anti-human CD146 is conjugated with the sequence consisting of SEQ ID NO: 1.

SEQ ID NO:9 is similar to SEQ ID NO:4 from the previous example, however the fluorescein modification at the 5′ end of the oligo is replaced with a modification less sensible to photobleaching, ATTO 488 fluorophore, and the spacer sequence between the biotin modification and the PCR primer called A has been removed (not necessary for the method, data no represented).

### Cell Staining

Cells in wells of the 48-well plate are washed with 1 ml of Cell Staining Buffer (Biolegend 420201). A mix of 20 μl of each antibody conjugated oligonucleotide (0.1 μg/μl of antibody), of 1 μl of Alexa Fluor 647 (AI647) anti-human CD146 Antibody (Biolegend 361013) and 2 μl of Brilliant Violet 421 (BV421) anti-human CD326 (Biolegend 324219) in a total of 200 μl of Cell Staining Buffer is added the cells. Cells are incubated for 30 min in the dark at 4° C. The antibody mix is removed before adding 1 ml of Cell Staining Buffer.

Anti-human CD146 antibody is specific to endothelial cells, including HUVEC cells, while anti-human CD326 antibody is specific to epithelial cells, including HEPG2 cells. It means that HUVEC cells are stained with A1647 fluorophore and the reporter sequence (SEQ ID NO:9) while HEPG2 cells are stained with BV421 fluorophore and the emitter sequence (SEQ ID NO:2).

### Reaction Mixture Assembly

A reaction mixture was made according to table 3 and is added to each well after removing of the Cell Staining Buffer.

TABLE 3
Reaction mixture

| | Final | Concentration | |
|---|---|---|---|
| | Concentration | unit | in μL |
| DNase/RNase-Free | | | 165.00 |
| Distilled Water | | | |
| (Invitrogen 10977023) | | | |
| Receptor (Seq ID 2) | 0.1 | μM | 2.00 |
| Quencher (Seq ID 4) | 0.5 | μM | 1.00 |
| Bst DNA Polymerase, | 0.2 | U/μl | 4.00 |
| Large Fragment | | | |
| (NEB M0275S) | | | |
| Nb.BbvCI (NEB R0631S) | 0.2 | U/μl | 4.00 |
| NEB 2 Buffer (B7002S) | 1 | x | 20.00 |
| dNTPs (NEB N0447L) | 0.2 | mM | 4.00 |
| Final Reaction Volume | | | 200.00 |

The plate is covered and is incubated under an inversed Nikon Ti-2 microscope equipped with 10× objective (MRD70170) and a filter wheel (T12-P-FWB-E), a quadriband dichroic and emission filter (Semrock, FF409/493/573/632-Di03-25x36 and FF01-432/515/595/730-25), a fluorescence source (Lumencor SPECTRA X) and a heating stage (Tokai Hit TP-TIZH26) set at 40° C. to reach at temperature of 37° C. at cells location.

During the 2 hours incubation, a 5 per 5 stitched image is taken every 10 min in each well, in brightfield, BV421, ATTO 488 and A1647 channels.

Therefore, HUVEC cells are observed in A1647 channel and HEPG2 in BV421 channel, while the reporter signal is observed in ATT0488 channel.

The fluorescent reporter (SEQ ID NO:9) present at the surface of the HUVEC cells is partially hybridized to a sequence carrying a quencher (SEQ ID NO:5), preventing the observation of fluorescence. The self-primed emitter (SEQ ID NO:2) carried by the HEPG2 cells releases nucleic acids. After diffusion and amplification, the released nucleic acids hybridized to a fluorescent reporter (SEQ ID NO:9). Then, the sequence carrying a quencher is released by polymerization of the released nucleic acids on the reporter, causing the appearance of the fluorescent signal.

The inventors observed 40 min after the start of the incubation the appearance of a signal in ATTO488, colocalized with the HUVEC staining in A1647 channel (FIG. 5).

### Example 4: Fresh Tissue Staining and Dissociation

Purified anti-human β2-microglobulin antibody (Biolegend 316302) is conjugated with streptavidin using Streptavidin Conjugation Kit (Abcam 102921). Conjugated antibodies are then mixed in 1× Tris Buffered Saline (TBS, VWR CAYM600232-500) with biotinylated oligonucleotides in 1:12 ratio and stored protected from light in a room with controlled temperature between 2° and 25° C. for over 12 h (over-night). After conjugation with streptavidin, anti-human β2-microglobulin antibody is mix in one tube with SEQ ID NO:1 and in another tube with SEQ ID NO:10.

Tissue slide is washed in a cell staining buffer (Biolegend 420201) before being incubation with a mix in cell staining buffer of anti-human β2-microglobulin antibodies conjugated with SEQ ID NO:1 at 0.5 μg/ml and SEQ ID NO:10 at 5 μg/ml, for 15-20 min at 4° C. The slide is then washed with cell staining buffer.

A reaction mixture is made according to table 4 and is added to the tissue slide after removing of the cell staining buffer. The volume can be adjusted to cover the surface of the tissue while respecting the final concentrations.

TABLE 4
Reaction mixture

| | Final | Concentration | |
|---|---|---|---|
| | Concentration | unit | in μL |
| DNase/RNase-Free | | | 166.00 |
| Distilled Water | | | |
| (Invitrogen 10977023) | | | |
| Receptor (Seq ID 3) | 0.1 | μM | 2.00 |
| Bst DNA Polymerase, | 0.2 | U/μl | 4.00 |
| Large Fragment | | | |
| (NEB M0275S) | | | |
| Nb.BbvCI (NEB R0631S) | 0.2 | U/μl | 4.00 |
| NEB 2 Buffer (B7002S) | 1 | x | 20.00 |
| dNTPs (NEB N0447L) | 0.2 | mM | 4.00 |
| Final Reaction Volume | | | 200.00 |

The tissue slide is place in a close environment to avoid evaporation and is incubated for 1 h at 37° C. The strategy for amplification and capture of emitter nucleic acids is detailed in FIG. 2.1.

After washing in HBSS (Thermo 14175053)+5% FBS (Thermo 16140071), the biopsy is minced as small as possible (around 1 mm).

The sample is then transferred to a tube with 200 μL of 20 mg/mL collagenase I (final: 2 mg/mL, Sigma C0130), 5 μL of 10 mg/mL Dnase I (final: 25 μg/mL, Sigma 11284932001), 80 μL of 50 mg/mL hyaluronidase (final: 2 mg/mL, Sigma H3506). The final volume is adjusted to 2 mL with HBSS. After 50 minutes of smooth agitation at 37° C. with regular up and down pipetting, the digested sample is filtered with a cell strainer (100 μm) and washed using TBS 1× (Thermo 14190169) with 1% HS (Thermo 26050088) and 2 mM EDTA (Thermo 15575020). Cells are resuspended in TBS 1X.

### Example 5: Spatially Resolved scRNA-Seq in Micro-Caqes

To get a spatially resolved scRNA-seq through the use thermo-actuable cages, single-cell isolation, barcoding and sequencing is performed in a chip using resuspended cells from example 4 of the present invention.

The overall method is as follows.

### Synthesis of the Thermo-Actuatable Hydrogel

An ene-functionalised poly(n-isopropyle acrylamide) is synthesized following the steps descripted in the supplementary information of D'Eramo L.; et al., Microsystems & Nanoengineering, 2018, 4, 17069, doi:10.1038.

The swelling properties of the obtained polymer, as a function of temperature, are evaluated in various aqueous solutions: pure water, phosphate saline buffer, at pH2 and at pH 9. The results are given in FIG. 7.

### Preparation of the First Substrate

The first substrate, made of polydimethylsiloxane (PDMS), is prepared by standard soft lithography techniques, containing microstructures and a chamber. The height of the structures and the chamber depends on the objective and can be a couple of a tenth of a micron up to 100 microns tall.

### Functionalization of the First Substrate

The PDMS substrate is exposed to Oxygen plasma for 50 s after being cleaned with isopropanol. Immediately following the surface activation, a solution of anhydrous toluene with a 3 vol % of mercaptopropyltrimethoxysilane (ABCR Gelest) is put in contact with the substrate for 3 h inside a reactor under nitrogen. Following the thiol-modification of the surface, the substrate is rinsed with toluene and finally dried with nitrogen flow.

Photo-Patterning of Hydrogel Films onto the First Substrate

Preformed functionalized pNIPAM (being ene-reactive) is spin-coated on the thiol-modified and micro-structured PDMS substrate with dithiol cross-linkers. A microvolume of a couple of 100 μL of a solution of butanol and methanol (V/V=1/1) containing the functionalized pNIPAM at a concentration between 3 and 15 wt. % and dithioerythritol (purchased from Sigma Aldrich, CAS number 3483-12-3) cross-linkers at a concentration between 3 and 10 wt. % is deposited onto the substrate. The conditions of spin-coating are fixed at an angular velocity varying between 500 rpm and 3000 rpm for a spinning time of 30 s. The spread films are dried by heat in a 90° C. oven for 5 minutes. The resulting layer thickness varies from a few tenth of a micron to 15 microns.

Chromium masks presenting numerous micro-structured cages are aligned with the design of the chamber and placed under UV lamp for deep UV exposure (8 watts, 250 nm wavelength). After exposure, free polymer chains are rinsed off by washing the substrate in an ultrapure water bath for 5 minutes. The hydrogel-patterned substrates are dried with nitrogen flow.

### Preparation of the Second Substrate

The second substrate used is a glass slide spotted with DNA strands (purchased from Agilent, referenced as an Agilent Microarray Format).

Presenting up to 1 million unique spots with different DNA strands grafted on it, this item provides different barcode on each spot. Each spot contains millions of DNA strands, each spot having a different barcode.

A localization system is integrated in the design of the array. Among the numerous unique spots, some of them carry a specific sequence for capture of fluorescently-labeled DNA oligonucleotides by hybridization (2 or more). They are placed so as to form multiple shapes comprising triangle, square and circle.

### Closure of the Device

Bonding between the first and the second substrates is achieved by using an O2 plasma treatment for 50 s. A protective layer is tapped onto the area of interest avoiding Oxygen plasma to be active there. After the termination of the exposure, the PDMS substrate is placed on top of the DNA array so that the region of interest faces the hydrogel structures. A curing step is applied, for 30 minutes at least, by storing the chip inside a 70° C. oven.

Preparation of the Chip for Specific Capture: Total scRNA-Seq

Oligonucleotides were design in silico and purchased from IDT, at 25 to 100 μM concentration in IDTE Buffer, pH 8.0, with standard desalting.

A solution of 100 mM Potassium Acetate, 30 mM HEPES, pH 7.5 and 10 μM of oligonucleotides Q, R and S is injected inside the microfluidic chamber at 40° C. (cages open) in order to hybridize the additional sequence of capture onto the seated DNA strands and to proceed to the localization step. The flow is stopped and the chip with the mixed oligonucleotides heated above 60° C. for 2 minutes, then incubated for 10 minutes at room temperature and rinsed with 1×SSC solution (Thermo Scientific #15413549) at 40° C. Oligonucleotides Q, R and S are of the following sequences:

Q: Pre_BclI_P5:
(SEQ ID NO: 12)
CATGCTTGATCAGACCACCGAGATCTAC
R:
Phos_Rd1_Nxt_UMI12_PolydT30VN:
(SEQ ID NO: 13)
5Phos/TCGTCGGCAGCGTCAGATGTGTATAAGAGACAGNNNNNNNNNN
NNTTTTTTTTTTTTTTTTTTTTTTTTTTTTTTVN
S:
Fluo_4.2_3Cy5Sp:
(SEQ ID NO: 14)
CATACTCGGTCTGCG/3Cy5Sp/

A brightfield and a fluorescent imaging (excitation at 650 nm and emission at 670 nm) of the main chamber is performed. An informatic identification of the DNA array spots and cage is performed. The identification is based on the superposition of both images. The brightfield image allows to identify the cages. The fluorescent image makes it possible to identify certain spots on which fluorescent DNA probes have been previously hybridized. The relative position of these circles define a unique location on the chip, allowing to map the original organization of the DNA array onto the superposed images. Finally, thanks to this identification, we can associate spot specific barcodes with each cage.

### Primer Synthesis.

A mix containing Sulfolobus DNA Polymerase IV (NEB #M0327S) and Hi-T4 ligase (NEB #M2622S) in Thermopol 1× (NEB #B9004S) supplemented with 1 mM ATP (NEB #P0756S) is injected at 40° C. and then incubated at 50° C. for 2 hours inside the chamber to polymerize and ligate barcoded primers. The inner volume is then rinsed with 1×SSC solution at 40° C. (Thermo Scientific #15413549).

### Capture of the Single Cells

A suspension of cells at a concentration of 10 million per ml with 1% BSA in TBS is prepared. Cells are injected inside the chip at 100 μl/h; the chip being heated up at 37° C. in order to open the cages. Once the cells are circulating around and above the cages, the flow is stopped, and the temperature is decreased to 20° C. to close the cages. Cells are trapped inside the cages.

To perform the lysis, a solution of 0.2% SDS (Sigma 71736) in PBS is injected inside the chamber through additional inlets that are not obstructed by the swollen hydrogel cages.

Cells' RNA strands are subsequently hybridized to the grafted sequence of capture as the aqueous phase around the cage is changed with PBS.

After a 10 minutes stop flow, a cleaning step is performed by opening the cage (increase temperature to 37° C.) and flowing PBS inside the chamber.

Barcoded cDNA Synthesis

A cleaning step is performed by opening the cage at 37° C. and flowing a volume of RT buffer (Thermo Scientific #EP0742) equivalent of 20 times the inner volume of the chamber through the chamber for 3 minutes.

After cleaning, a mix including 10 U/μl of Reverse Transcriptase (Thermo Scientific #EP0742), 0.5 mM dNTPs (NEB #N0447S) and 1U/μl of RNase Inhibitor (Thermo Scientific #11581505) in 1× RT buffer is injected inside the chamber, followed by an immobilization of the flow and reverse transcription of the captured strands for 2 h at 50° C. The enzyme is immediately flushed with a 1×SSC solution to stop reverse transcription.

A second mix containing 2U/μl of exonuclease I in 1× Exonuclease I Reaction Buffer (NEB #M0293S) is injected and incubated at 37° C. for 30 minutes. The enzyme is immediately flushed with a 1× SSC solution to stop the reaction.

DNase/RNase-Free Distilled Water (Invitrogen #10977023) is injected in the chamber and heated up to 98° C. to dehybridize synthesized cDNA. The inner volume is then collected by flowing additional water.

10 μL of NEB2 Buffer (NEB #M0212L) and 10 μL of 10 μM of oligonucleotide T is added to 65 μL of the recovered sample.

The solution is incubated at 95° C. for 2 minutes and transferred immediately to ice. Then, 8 μL of 10 nM dNTPs and 7 μL of Klenow exo-(NEB #M0212L) is added.

The mix is placed in a thermocycler pre-chilled at 4° C., the temperature ramped slowly to 37° C., and hold for 30 minutes to perform the synthesis of the second strand.

cDNAs are then purified using SPRIselect magnetic beads (Beckman #B23317) following the manufacturer's instructions.

A 2-step PCR amplification with an elongation of 30 seconds is then performed for 15 cycles on the purified sample using primers U and V and Q5 High-Fidelity DNA Polymerase (NEB #M0491).

### Oligonucleotides U and V are of the Following Sequences:

U: P5:
(SEQ ID NO: 15)
AATGATACGGCGACCACCGAGATCTACAC
V: P7_i05_Rd2_Nxt:
(SEQ ID NO: 16)
CAAGCAGAAGACGGCATACGAGATCCAGGAAGGTCTCGTGGGCTCGGAG
ATGTGTATAAGAGACAG.

The PCR product is purified using SPRIselect magnetic beads (Beckman #B23317) and quantified before being sequenced using an Illumina sequencing platform.

The demultiplexing of the sequencing data is performed by retrieving for each read the barcodes from position 1 to 15 of Index1 reads, the UMI from position 1 to 12 of Read1 reads and the RNA transcript starting from position 1 of Read2 reads.

By replacing 10% of oligonucleotide Q with a similar oligonucleotide but carrying at the 3′ end the capture sequence of the emitter nucleic acids (SEQ ID NO:3) instead of the polyT tail, after cell lysis in the thermo-actuable cages, emitter nucleic acids hitherto present on the cell surface, hybridize with the cage-specific primers and are associated after DNA synthesis with a cage barcode as for the mRNA.

An amplification is performed with a subpart of the cDNA solution using a supplementary primer to U, which is V comprising at its 3′ end the amplification sequence of the emitter nucleic acids, represented by SEQ ID NO:11.

After sequencing, identification sequences of the emitter nucleic acids of a cell can be link to mRNA transcript of the same cell thanks to the cage barcode. Presence of identical identification sequences and their relative amount is used to rebuild the cellular neighborhood.

### Example 6: Spatially Resolved scRNA-Seq on Drop-Seq Platform

To get a spatially resolved scRNA-seq, Drop-seq is performed as described in (Macosko, E. Z. et al. Cell 161, 1202-1214 (2015)) with modifications using resuspend cells from example 4. 10% of the oligonucleotide on the hydrogel beads are carrying at their 3′ end the capture sequence of the emitter nucleic acids (SEQ ID NO:3) replacing the polyT tail. Emitter nucleic acids are thus associated with the same droplet barcode as the mRNA.

cDNAs are separated and amplified as described in Stoeckius, M et al. Simultaneous epitope and transcriptome measurement in single cells. Nat Methods 14, 865-868 (2017) with modifications. Barcoded emitter nucleic acids are amplified using supplementary primers comprising at their 3′ end the amplification sequence of the emitter nucleic acids, represented by SEQ ID NO:11. After sequencing, identification sequences of the emitter nucleic acids of a cell can be link to mRNA transcript of the same cell thanks to the cage barcode. Presence of identical identification sequences and their relative amount is used to rebuild the cellular neighborhood.

1. A method for labelling individual cells or organelles within a biological sample with an identification nucleic acid sequence, the method comprising:
a) providing a first set of nucleic acids (“Emitter nucleic acids”) wherein each nucleic acid molecule comprises an amplification sequence, an identification sequence, and a capture sequence;
b) providing a second set of nucleic acids (“Receptor nucleic acids”), wherein each Receptor nucleic acid is coupled, covalently or non-covalently, to a ligand of a cell target or organelle target, wherein the Receptor nucleic acid comprises i) an amplification sequence matching all or part of the amplification sequence of the set of Emitter nucleic acids, or a complement thereof, or ii) a capture sequence matching all or part of the capture sequence of the set of Emitter nucleic acids, or a complement thereof,
c) contacting the sets of Emitter nucleic acids and Receptor nucleic acids, in solution, with a biological sample so as to label the individual cells or organelles within the biological sample at least with the Receptor nucleic acids;
d) releasing multiple nucleic acid molecules that are copies of the region of the Emitter nucleic acids that comprises the amplification sequence, the identification sequence, and the capture sequence, or the reverse complement thereof (“Emitted nucleic acids”), and hybridizing the Emitted nucleic acids to Receptor nucleic acids;
e) dissociating the biological sample and recovering individualized cells or organelles, wherein at least a sub-population of the individualized cells or organelles is labelled with the Emitted nucleic acids.

2. The method according to claim 1, wherein each Emitter nucleic acid is bound to a ligand of a cell target or organelle target.

3. The method according to claim 2, wherein at step c), the biological sample is rinsed after contacting with the sets of Emitter nucleic acids and Receptor nucleic acids.

4. The method according to claim 1, wherein the Emitter nucleic acids additionally comprise a sequence complementary to a nicking site and in step c) production of Emitted nucleic acids is performed by isothermal amplification by nicking and polymerization, catalyzed by a nicking endonuclease and a polymerase with strand displacement activity.

5. The method according to claim 1, wherein the set of Emitter nucleic acids is provided in the form of groups of Emitter nucleic acids wherein all Emitter nucleic acids of a group comprise an identical identification sequence.

6. The method according to claim 5, wherein a group of Emitter nucleic acids comprises:
(a) concatemers of Emitter nucleic acids generated by rolling-circle replication, wherein each concatemer comprises identical Emitter nucleic acids; or
(b) beads carrying Emitter nucleic acids that comprise an identical identification sequence.

7. The method according to claim 5, wherein at step d), the Emitter nucleic acids of a group are cut by a restriction endonuclease to release multiple copies of the Emitted nucleic acids.

8. The method according to claim 1, wherein the Receptor nucleic acids further comprise a restriction site or a cleavage site.

9. The method according to claim 1, wherein the cell target or organelle target is a target ubiquitously present at the surface or inside all or most of the cells or any organelles of the cells in the biological sample.

10. The method according to claim 1, wherein an image of the biological sample is taken by microscopy before step d) or before step e).

11. A method of mapping and sequencing individual cells or organelles of a biological sample, the method comprising:
a) Providing individualized cells labelled with (i) a nucleic acid that comprises an amplification sequence, an identification sequence, and a capture sequence, or (ii) the reverse complement thereof (i), as obtainable by the method of claim 1;
b) Trapping the individualized cells or organelles labelled with said nucleic acid or the reverse complement thereof in a compartment, wherein the compartment comprises a compartment-specific nucleic acid and at least one of the following sequences for nucleic acid labeling and further sequencing: hybridization site, ligation site or recombination site;
c) Optionally analyzing captured cells, organelles and/or molecules they secrete, using optical detection;
d) Lysing trapped cells, or cells and organelles, thereby releasing nucleic acids from the cells or organelles in the compartments;
e) Associating i) the compartment-specific sequence with ii) the nucleic acids released from the cells or organelles in the compartments and iii) the nucleic acids comprising the amplification sequence, the identification sequence, and the capture sequence, or the reverse complement thereof,
f) Recovering the nucleic acids produced at step e) from the compartments and sequencing recovered nucleic acids; and
g) Defining nucleic acids comprising the same compartment-specific sequence as originating from the same single cell, and mapping the position of the single cell originally on the biological sample based on the identification sequence(s), or reverse complementary sequence thereof, contained in the nucleic acids produced at step e), thereby combining mapping and sequencing information of the individual cells of the biological sample;
h) Mapping of the position of the single cell originally on the biological sample is performed by determining the relative proportions of identical identification sequence(s), or reverse complementary sequence thereof, contained the nucleic acids produced at step e) to estimate the distance between cells, on the principle of triangulation; and
i) Optionally, mapping the sequencing information back onto an image from microscopy of the biological sample, taken before dissociation.

12. The method of mapping and sequencing according to claim 11, wherein the compartments are droplets, an hydrogel matrix, microfabricated chambers separated by pneumatic valves, microfabricated wells, actuatable hydrogel cages or microplate wells.

13. The method of mapping and sequencing according to claim 11, wherein the compartment-specific nucleic acid comprised in the compartment is DNA.

14. The method of mapping and sequencing according to claim 11, wherein step e) comprises either:
i) hybridizing the compartment-specific nucleic acid by complementarity to the released nucleic acids from the cell or organelle;
ii) hybridizing the compartment-specific nucleic acid by complementarity to the released nucleic acids from the cell or organelle and extending the compartment-specific nucleic acid hybridized to the released nucleic acids using a DNA polymerase to create the complementary strand of the released nucleic acids with an associated compartment-specific sequence;
iii) hybridizing the compartment-specific nucleic acid by complementarity to the 3′-end of cDNAs produced by reverse transcription of RNAs from the cell or organelle and extending the cDNA hybridized to the compartment-specific nucleic acid using a DNA polymerase to create the complementary strand of the compartment-specific nucleic acid with an associated compartment-specific sequence;
iv) ligating the compartment-specific nucleic acid to the DNA present in the compartment; or
v) recombining the compartment-specific nucleic acid with the DNA present in the compartment.

15. The method of mapping and sequencing according to claim 11, wherein:
the compartment-specific nucleic acids comprise a primer sequence complementary to all or part of the amplification or capture sequence present in the nucleic acid that comprises the amplification sequence, the identification sequence, and the capture sequence, or in the reverse complementary nucleic acid thereof;
step e) additionally comprises hybridizing the compartment-specific nucleic acids to all or part of the amplification or capture sequence present in the nucleic acid that comprises the amplification sequence, the identification sequence, and the capture sequence, or in the reverse complementary nucleic acid thereof, and extending one or both hybridized DNA strands using a DNA polymerase to create a DNA molecule comprising both the identification sequence, or it's reverse complement, and the compartment-specific sequence, or its complement, or alternatively, instead of the hybridization and extension steps, step e) additionally comprises ligating the compartment-specific nucleic acids to all or part of the amplification or capture sequence present in the nucleic acid, or recombining the compartment-specific nucleic acids to all or part of the amplification or capture sequence present in the nucleic acid.

16. A kit comprising:
a) Emitter nucleic acids wherein each emitter nucleic acid comprises an amplification sequence, an identification sequence, and a capture sequence;
b) Receptor nucleic acids wherein each receptor nucleic acid comprises i) an amplification sequence matching all or part of the amplification sequence of the set of Emitter nucleic acids, or a complement thereof, or ii) a capture sequence matching all or part of the capture sequence of the set of Emitter nucleic acids, or a complement thereof;
c) Ligands of a cell target or organelle target;
d) Optionally a nicking endonuclease and a polymerase with strand displacement activity.


SEQUENCE LISTING (16 sequences)

SEQ ID NO: 1 - DNA, 16 bp, synthetic construct
  source 1..16: mol_type = other DNA
  modified_base 1: mod_base = OTHER; note = 5' 6-FAM (Fluorescein)
  modified_base 16: mod_base = OTHER; note = 3' biotin
cacagggtgatcaggt

SEQ ID NO: 2 - DNA, 98 bp, synthetic construct
  source 1..98: mol_type = other DNA
  unsure 31..42
  modified_base 84: mod_base = OTHER; note = Int Biotin dT
aacaccaaacccttctaaagcccaaacctcnnnnnnnnnnnnaagcgatctgttaccaagccgtcctcagcaggattagagtttttctctaatcctgc

SEQ ID NO: 3 - DNA, 30 bp, synthetic construct
  source 1..30: mol_type = other DNA
  modified_base 1: mod_base = OTHER; note = 5' 6-FAM (Fluorescein)
  modified_base 30: mod_base = OTHER; note = 3' biotin
aacaccaaacccttctaaagcccaaacctc

SEQ ID NO: 4 - DNA, 74 bp, synthetic construct
  source 1..74: mol_type = other DNA
acacgttgatctagtcgccaccaacaccaaacccttctaaagcccaaacctccattcgttccgctcgcaacaat

SEQ ID NO: 5 - DNA, 22 bp, synthetic construct
  source 1..22: mol_type = other DNA
  modified_base 22: mod_base = OTHER; note = 3IABkFQ
ggtggcgactagatcaacgtgt

SEQ ID NO: 6 - DNA, 17 bp, synthetic construct
  source 1..17: mol_type = other DNA
acggcttggtaacagat

SEQ ID NO: 7 - DNA, 20 bp, synthetic construct
  source 1..20: mol_type = other DNA
aacaccaaacccttctaaag

SEQ ID NO: 8 - DNA, 22 bp, synthetic construct
  source 1..22: mol_type = other DNA
acacgttgatctagtcgccacc

SEQ ID NO: 9 - DNA, 52 bp, synthetic construct
  source 1..52: mol_type = other DNA
  modified_base 52: mod_base = OTHER; note = 3' biotin
  modified_base 1: mod_base = OTHER; note = ATTO 488 fluorophore
acacgttgatctagtcgccaccaacaccaaacccttctaaagcccaaacctc

SEQ ID NO: 10 - DNA, 30 bp, synthetic construct; identical to SEQ ID NO: 3
  source 1..30: mol_type = other DNA
  modified_base 30: mod_base = OTHER; note = 3' biotin

SEQ ID NO: 11 - DNA, 22 bp, synthetic construct
  source 1..22: mol_type = other DNA
acggcttggtaacagatcgctt

SEQ ID NO: 12 - DNA, 28 bp, synthetic construct
  source 1..28: mol_type = other DNA
catgcttgatcagaccaccgagatctac

SEQ ID NO: 13 - DNA, 77 bp, synthetic construct
  source 1..77: mol_type = other DNA
tcgtcggcagcgtcagatgtgtataagagacagnnnnnnnnnnnnttttttttttttttttttttttttttttttvn

SEQ ID NO: 14 - DNA, 15 bp, synthetic construct
  source 1..15: mol_type = other DNA
catactcggtctgcg

SEQ ID NO: 15 - DNA, 29 bp, synthetic construct
  source 1..29: mol_type = other DNA
aatgatacggcgaccaccgagatctacac

SEQ ID NO: 16 - DNA, 66 bp, synthetic construct
  source 1..66: mol_type = other DNA
caagcagaagacggcatacgagatccaggaaggtctcgtgggctcggagatgtgtataagagacag